(19) United States
(12) Patent Application Publication
(10) Pub. No.: US 2025/0235424 A1
(43) Pub. Date: Jul. 24, 2025

(54) PHARMACEUTICAL COMPOSITION CONTAINING DOCETAXEL OR PHARMACEUTICALLY ACCEPTABLE SALT THEREOF AND PREPARATION METHOD THEREFOR

(71) Applicants: WEBIOTREE CO., LTD, Seoul (KR); CNPHARM CO., LTD., Seoul (KR)

(72) Inventors: Ho Jun KIM, Seoul (KR); Ki Yeok KIM, Seoul (KR); Geun Woo JIN, Seoul (KR)

(73) Assignees: WEBIOTREE CO., LTD, Seoul (KR); CNPHARM CO., LTD., Seoul (KR)

(21) Appl. No.: 18/844,613

(22) PCT Filed: Dec. 20, 2022
(86) PCT No.: PCT/KR2022/020875

§ 371 Date: Sep. 6, 2024

(30) Foreign Application Priority Data
Mar. 8, 2022 (KR) 10-2022-0029687

(51) Int. Cl. A61K 31/337; A61K 9/16; A61P 35/00

(52) U.S. Cl. CPC A61K 31/337; A61K 9/1611; A61K 9/1617; A61K 9/1635; A61K 9/1641; A61K 9/1652; A61K 9/1682; A61P 35/00

(57) ABSTRACT

The present invention relates to a pharmaceutical composition containing docetaxel or a pharmaceutically acceptable salt thereof and a method for preparing the same, which can improve the low body absorption rate of docetaxel.

### TECHNICAL FIELD

The present invention relates to a pharmaceutical composition containing docetaxel or a pharmaceutically acceptable salt thereof and a method for preparing the same, which can improve the low body absorption rate of docetaxel.

### BACKGROUND ART

In developing oral dosage forms of a poorly soluble drug, enhancing the dissolution and solubility to enhance the bioavailability of the drug is becoming increasingly important. In particular, a typical example is docetaxel, which is known to have an anticancer effect but does not dissolve enough to exert an efficacious drug effect in vivo. Due to the structure, the drug is difficult to be absorbed in a patient, so its use is limited. While various studies have attempted to solve this problem, enhancing the solubility and dissolution of the drug by solubilizing the drug acts as the most critical factor in drug absorption.

In general, a solid dispersion technology for solubilization has been employed, and a method of granulating a crystalline drug into an amorphous drug using a poorly soluble drug, an ionic or non-ionic polymer, a surfactant, or the like has been used. Accordingly, Korean Patent Publication No. 10-2011-0020779 and the like disclose compositions for improving the effectiveness of docetaxel.

Even in the disclosed contents, attempts have been made to provide docetaxel with improved effects by using a polymeric material. However, docetaxel is merely injected through intravenous injection, and the disclosures did not show any enhanced solubility enough to be administered orally. That is, there is still a problem in that a significant effect has not been achieved to the extent that the pharmacological effect of docetaxel can actually be shown.

Patent Document: Korean Patent Publication No. 10-2011-0020779

### DISCLOSURE OF THE INVENTION

### Technical Problem

An object of the present invention is to provide a pharmaceutical composition containing docetaxel or a pharmaceutically acceptable salt thereof and a method for preparing the same, which can improve the low body absorption rate of docetaxel.

In addition, another object of the present invention is to provide a pharmaceutical composition capable of stably delivering a drug to the intestine by slowing the crystallization of the drug in the body in order to improve the absorption rate in the body and minimizing the effects of body fluids, such as gastric acid or bile acid, upon oral administration, and a method for preparing the same.

### Technical Solution

The present invention provides a pharmaceutical composition comprising: docetaxel or a pharmaceutically acceptable salt thereof; and one or more compounds selected from among a polyvinyl pyrrolidone-based compound, a cellulose-based compound, a poloxamer-based compound, a polyethylene glycol-based compound, an alginic acid-based compound, polygamma glutamic acid and sodium dodecyl sulfate.

In addition, the present invention provides a method for preparing a pharmaceutical composition, the method comprising the steps of: preparing a first dissolved product by dissolving one or more compounds selected from among a polyvinyl pyrrolidone-based compound, a cellulose-based compound, a poloxamer-based compound, a polyethylene glycol-based compound, an alginic acid-based compound, polygamma glutamic acid and sodium dodecyl sulfate, polygamma glutamic acid and sodium dodecyl sulfate in anhydrous ethanol; and preparing a second dissolved product by adding and stirring docetaxel or a pharmaceutically acceptable salt thereof to the first dissolved product.

### Advantageous Effects

The present invention allows a drug with a low bioabsorption rate, such as docetaxel or a pharmaceutically acceptable salt thereof, of an element of the composition, to have a significant blood concentration and bioavailability to the extent that a desired pharmacological effect could be achieved.

In addition, the present invention provides a pharmaceutical composition with the effect of enabling stable delivery of a drug to the intestine by slowing the crystallization of the drug in the body in order to improve the absorption rate in vivo and minimizing the effects of body fluids, such as gastric acid or bile acid, upon oral administration.

In addition, in the present invention, a polymer material for a poorly soluble drug, such as docetaxel, is used to form a drug-matrix composition so that the low dispersibility and blood concentration maintenance effect, which are problems of the poorly soluble drug, could be improved. Therefore, the present a invention provides composition with excellent bioavailability.

In addition, the present invention provides a method for preparing a composition capable of solving problems due to the poor solubility of docetaxel or a pharmaceutically acceptable salt thereof.

### BRIEF DESCRIPTION OF THE DRAWINGS

FIG. 1 is the graph of plasma levels over time obtained by orally administering the compositions of Examples 1 to 10 to rats.

FIG. 2 is the graph of the AUC results obtained by orally administering the compositions of Examples 1 to 10 to rats.

FIG. 3 is the graph of plasma levels over time obtained by orally administering the compositions of Examples 23 to 33 to rats.

FIG. 4 is the graph of the AUC results obtained by orally administering the compositions of Examples 23 to 33 to rats.

FIG. 5 is the graph of plasma levels over time obtained by orally administering the compositions of Examples 68 to 75 to rats.

FIG. 6 is the graph of plasma levels over time obtained by orally administering the compositions of Examples 76 to 82 to rats.

FIG. 7 is the graph of the AUC results obtained by orally administering the compositions of Examples 68 to 82 to rats.

FIG. 8 is the graph of plasma levels over time obtained by orally administering the compositions of Example 83 and Comparative example 1 to rats.

FIG. 9 is the graph of the AUC results obtained by orally administering the compositions of Example 83 and Comparative example 1 to rats.

FIG. 10 is the graph of body weight change by administration group after administering the compositions of Example 78, Comparative Example 2, and Control Example 1 to mice (Data are expressed as Mean±S.D. The day of the first administration was designated as day 0).

FIG. 11 is the graph of tumor size change by administration group after administering the compositions of Example 78, Comparative Example 2, and Control Example 1 to mice (Data are expressed as Mean±S.D.).

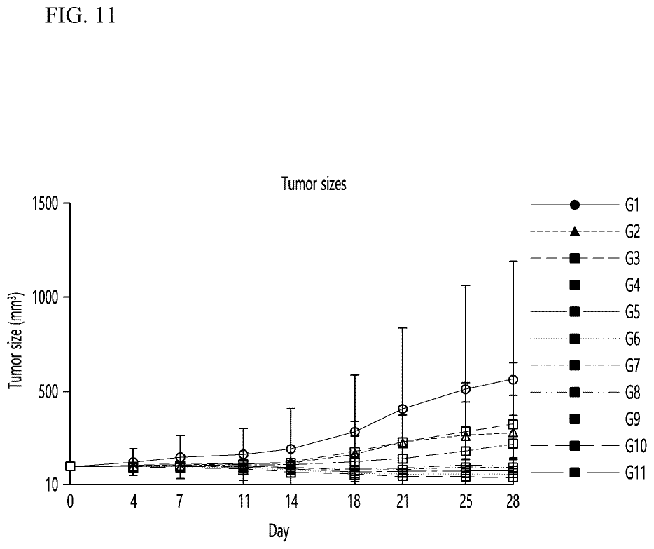

FIG. 12 is the graph of tumor weight change by administration group after administering the compositions of Example 78, Comparative Example 2, and Control Example 1 to mice (Data are expressed as Mean±S.D. The day of the first administration was designated as day 0. **/* A significant difference at p<0.01/p<0.05 level compared to the G1).

### MODE FOR CARRYING OUT THE INVENTION

The present invention relates to a composition capable of improving the bioavailability of a drug in vivo and to a pharmaceutical composition comprising: docetaxel or a pharmaceutically acceptable salt thereof; and one or more of a polyvinyl pyrrolidone-based compound, a cellulose-based compound, a poloxamer-based compound, a polyethylene glycol-based compound, an alginic acid-based compound, polygamma glutamic acid and sodium dodecyl sulfate.

### MODE FOR CARRYING OUT THE INVENTION

In the present invention, a pharmaceutically acceptable salt refers to a salt typically used in the pharmaceutical industry. For example, a pharmaceutically acceptable salt may include inorganic ion salts made of calcium, potassium, sodium, magnesium, and the like; inorganic acid salts made of hydrochloric acid, nitric acid, phosphoric acid, bromic acid, iodic acid, perchloric acid, sulfuric acid, and the like; organic salts acid made of acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like; sulfonic acid salts made of methanesulfonic acid, ethanesulfonic acid, benzenesulfonic acid, p-toluenesulfonic acid, naphthalenesulfonic acid, and the like; amino acid salts made of glycine, arginine, lysine, and the like; and amine salts made of trimethylamine, triethylamine, ammonia, pyridine, picoline, and the like. However, types of salts meant in the present invention are not limited to these listed salts.

In the present invention, it is confirmed that when a pharmaceutical composition is prepared by combining docetaxel or a pharmaceutically acceptable salt thereof and one or more compounds selected from among a polyvinyl pyrrolidone-based compound, a cellulose-based compound, a poloxamer-based compound, a polyethylene glycol-based compound, an alginic acid-based compound, polygamma glutamic acid and sodium dodecyl sulfate, the pharmaceutical composition can have better bioavailability than a simple combination of docetaxel and a polyvinyl pyrrolidone-based compound.

In the present invention, it is confirmed that in order to generate a higher pharmacological effect of a poorly soluble drug such as docetaxel, in addition to combining the drug with one or more compounds selected from a polyvinyl pyrrolidone-based compound, a cellulose-based compound, a poloxamer-based compound, and a polyethylene glycol-based compound, loading a polymer compound having a crystallization-inhibitory effect or a swellable function with docetaxel or a pharmaceutically acceptable salt thereof can increase the drug's solubility in the body by delaying the crystallization time of the drug and increase the blood concentration of the drug.

Specifically, the polyvinyl pyrrolidone-based compound may be one or more selected from the group consisting of polyvinylpyrrolidone K10 (MW 8000-10,000), polyvinylpyrrolidone K12 (MW 11,000-12,000), polyvinylpyrrolidone K15 (MW 14,000-18,000), polyvinylpyrrolidone K17 (MW 14,000-18,000), polyvinylpyrrolidone K18 (MW 14,000-18,000), polyvinylpyrrolidone K25 (MW 2,000-25,000), polyvinylpyrrolidone K30 (MW3,000-40,000), polyvinylpyrrolidone K60 (MW 5,000-60,000), and polyvinylpyrrolidone K90 (MW 8,000-90,000). In the above, MW stands for molecular weight and means weight average molecular weight.

In addition, the cellulose-based compound may be one or more selected from the group consisting of hydroxypropyl methylcellulose (HPMC), hydroxyethyl cellulose, hydroxypropyl cellulose (HPC), carboxymethylcellulose (CMC), ethylcellulose (EC), methylcellulose (MC) and cellulose acetate (CA). The above-mentioned cellulose-based compound may have a weight average molecular weight of 5,000 to 500,000.

In addition, the poloxamer-based compound may be one or more selected from the group consisting of poloxamer 101, poloxamer 105, poloxamer 105 benzoate, poloxamer 108, poloxamer 122, poloxamer 123, poloxamer 124, poloxamer 181, poloxamer 182, poloxamer 182 dibenzoate, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, and poloxamer 407 may be more preferred. The above-mentioned poloxamer-based compound may have a weight average molecular weight of 5,000 to 500,000.

In addition, the polyethylene glycol-based compound may be one or more selected from the group consisting of polyethylene glycol 200, polyethylene glycol 300, polyethylene glycol 400, polyethylene glycol 500, polyethylene glycol 1000, polyethylene glycol 1400, polyethylene glycol 1500, polyethylene glycol 4000, polyethylene glycol 8000, polyethylene glycol 10000, and methoxy polyethylene glycol 550. The above-mentioned polyethylene glycol-based compound may have a weight average molecular weight of 5,000 to 500,000.

In addition, the alginic acid-based compound may be alginic acid or alginic acid salt, and the alginic acid salt may be one or more selected from among sodium alginate (Na-alginate), potassium alginate, calcium alginate, ammonium alginate, and triethanol ammonium alginate.

The pharmaceutical composition of the present invention may further contain one or more compounds selected from the group consisting of magnesium oxide (MgO), bentonite mineral, hydrotalcite, and magnesium hydroxide. When one or more selected from among magnesium oxide (MgO), bentonite mineral, hydrotalcite, and magnesium hydroxide are further included, the absorption rate of docetaxel in the body can be increased to further improve the blood solubility, and thus the pharmaceutical composition can have substantially excellent bioavailability. In this regard, further containing one or more compounds selected from the group consisting of magnesium oxide (MgO), bentonite mineral, hydrotalcite, and magnesium hydroxide is preferable. The bentonite mineral may, more preferably, contain by or more 90% weight of montmorillonite.

The pharmaceutical composition the of present invention may include one or more combinations selected from combination docetaxel or among: a 1st comprising a pharmaceutically acceptable salt thereof (hereinafter, referred to as ‘docetaxel’) and a polyvinyl pyrrolidone-based compound; a 2nd combination comprising docetaxel and a cellulose-based compound; a 3rd combination comprising docetaxel and a poloxamer-based compound; a 4th combination comprising docetaxel and a polyethylene glycol-based compound; a 5th combination comprising docetaxel and an alginic acid-based compound; a 6th combination comprising docetaxel and polygamma glutamic acid; a 7th combination comprising docetaxel and sodium dodecyl sulfate; an 8th combination comprising docetaxel, a polyvinyl pyrrolidone-based compound, and a cellulose-based compound; a 9th combination comprising docetaxel, a polyvinyl pyrrolidone-based compound, and a poloxamer-based compound; a 10th combination comprising docetaxel, a polyvinyl pyrrolidone-based compound, and a polyethylene glycol-based compound; an 11th combination comprising docetaxel, a polyvinyl pyrrolidone-based compound, and an alginic acid-based compound; a 12th combination comprising docetaxel, a polyvinyl pyrrolidone-based compound, and polygamma glutamic acid; a 13th combination comprising docetaxel, a polyvinyl pyrrolidone-based compound, and sodium dodecyl sulfate; a 14th combination comprising docetaxel, a cellulose-based compound, and a poloxamer-based compound; a 15th combination comprising docetaxel, a cellulose-based compound, and a polyethylene glycol-based compound; a 16th combination comprising docetaxel, a cellulose-based compound, and an alginic acid-based compound; a 17th combination comprising docetaxel, a cellulose-based compound and polygamma glutamic acid; an 18th combination comprising docetaxel, a cellulose-based compound, and sodium dodecyl sulfate; a 19th combination comprising docetaxel, a poloxamer-based compound, and a polyethylene glycol-based compound; a 20th combination comprising docetaxel, a poloxamer-based compound, and an alginic acid-based compound; a 21st combination comprising docetaxel, a poloxamer-based compound, and polygamma glutamic acid; a 22nd combination comprising docetaxel, a poloxamer-based compound and sodium dodecyl sulfate; a 23rd combination comprising docetaxel, a polyethylene glycol-based compound, and an alginic acid-based compound; a 24th combination comprising docetaxel, a polyethylene glycol compound, and polygamma glutamic acid; a 25th combination comprising docetaxel, a polyethylene glycol-based compound, and sodium dodecyl sulfate; a 26th combination comprising docetaxel, an alginic acid-based compound, and polygamma glutamic acid; a 27th combination comprising docetaxel, polygamma glutamic acid, and sodium dodecyl sulfate; a 28th combination comprising docetaxel, polygamma glutamic acid, and sodium dodecyl sulfate; a 29th combination comprising docetaxel, a polyvinyl pyrrolidone-based compound, a cellulose-based compound, and a poloxamer-based compound; a 30th combination comprising docetaxel, a polyvinyl pyrrolidone-based compound, a cellulose-based compound, and a polyethylene glycol-based compound; a 31st combination comprising docetaxel, a polyvinyl pyrrolidone-based compound, a cellulose-based compound, and an alginic acid-based compound; a 32nd combination comprising docetaxel, a polyvinyl pyrrolidone-based compound, a cellulose-based compound, and poly-gamma glutamic acid; a 33rd combination comprising docetaxel, a polyvinyl pyrrolidone-based compound, a cellulose-based compound, and sodium dodecyl sulfate; a 34th combination comprising docetaxel, a polyvinyl pyrrolidone-based compound, a poloxamer-based compound, and a polyethylene glycol-based compound; a 35th combination comprising docetaxel, a polyvinyl pyrrolidone-based compound, a poloxamer-based compound, and an alginic acid-based compound; a 36th combination comprising docetaxel, a polyvinyl pyrrolidone-based compound, a poloxamer-based compound, and sodium dodecyl sulfate; a 37th combination comprising docetaxel, a polyvinyl pyrrolidone-based compound, a polyethylene glycol-based compound, and an alginic acid-based compound; a 38th combination comprising docetaxel, a polyvinyl pyrrolidone-based compound, a polyethylene glycol-based compound, and polygamma glutamic acid; a 38th combination comprising docetaxel, a polyvinyl pyrrolidone-based compound, a polyethylene glycol-based compound, and sodium dodecyl sulfate; a 39th combination comprising docetaxel, a polyvinyl pyrrolidone-based compound, an alginic acid-based compound, and polygamma glutamic acid; a 40th combination comprising docetaxel, a polyvinyl pyrrolidone-based compound, an alginic acid-based compound, and sodium dodecyl sulfate; a 41st combination comprising docetaxel, a polyvinyl pyrrolidone-based compound, polygamma glutamic acid, and sodium dodecyl sulfate; a 42nd combination comprising docetaxel, a cellulose-based compound, a poloxamer-based compound, and an alginic acid-based compound; a 43rd combination comprising docetaxel, a cellulose-based compound, a poloxamer-based compound, and polygamma glutamic acid; a 44th combination comprising docetaxel, a cellulose-based compound, a poloxamer-based compound, and a polyethylene glycol-based compound; a 45th combination comprising docetaxel, a cellulose-based compound, a poloxamer-based compound, and polygamma glutamic acid; a 46th combination comprising docetaxel, a cellulose-based compound, a poloxamer-based compound, and an alginic acid-based compound; a 47th combination comprising docetaxel, a cellulose-based compound, a poloxamer-based compound, and sodium dodecyl sulfate; a 48th combination comprising docetaxel, a cellulose-based compound, a polyethylene glycol-based compound, and an alginic acid-based compound; a 49th combination comprising docetaxel, a cellulose-based compound, a polyethylene glycol-based compound, and polygamma glutamic acid; a 50th combination comprising docetaxel, a cellulose-based compound, a polyethylene glycol-based compound, and sodium dodecyl sulfate; a 51st combination comprising docetaxel, a cellulose-based compound, an alginic acid-based compound, and sodium dodecyl sulfate; a 52nd combination comprising docetaxel, a cellulose-based compound, an alginic acid-based compound, and polygamma glutamic acid; a 53rd combination comprising docetaxel, a cellulose-based compound, polygamma glutamic acid, and sodium dodecyl sulfate; a 54th combination comprising docetaxel, a poloxamer-based compound, a polyethylene glycol-based compound, and an alginic acid-based compound; a 55th combination comprising docetaxel, a poloxamer-based compound, a polyethylene glycol-based compound, and polygamma glutamic acid; a 56th combination comprising docetaxel, a poloxamer-based compound, a polyethylene glycol-based compound, and an alginic acid-based compound; a 57th combination comprising docetaxel, a poloxamer-based compound, a polyethylene glycol-based compound, and sodium dodecyl sulfate; a 58th combination comprising docetaxel, a poloxamer-based compound, an alginic acid-based compound, and polygamma glutamic acid; a 59th combination comprising docetaxel, a poloxamer-based compound, an alginic acid-based compound, and sodium dodecyl sulfate; a 60th combination comprising docetaxel, a poloxamer-based compound, polygamma glutamic acid, and sodium dodecyl combination comprising docetaxel, a polyvinyl pyrrolidone-based compound, a cellulose-based compound, a poloxamer-based compound, and a polyethylene glycol-based compound; a 62nd combination comprising docetaxel, a polyvinyl pyrrolidone-based compound, a cellulose-based compound, a poloxamer-based compound, and an alginic acid-based compound; a 63rd combination comprising docetaxel, a polyvinyl pyrrolidone-based compound, a cellulose-based compound, a poloxamer-based compound, and polygamma glutamic acid; a 64th combination comprising docetaxel, a polyvinyl pyrrolidone-based compound, a cellulose-based compound, a poloxamer-based compound, and sodium dodecyl sulfate; a 65th combination comprising docetaxel, a polyvinyl pyrrolidone-based compound, a cellulose-based compound, a polyethylene glycol-based compound, and an alginic acid-based compound; a 66th combination comprising docetaxel, a polyvinyl pyrrolidone-based compound, a cellulose-based compound, a polyethylene glycol-based compound, and polygamma glutamic acid; a 67th combination comprising docetaxel, a polyvinyl pyrrolidone-based compound, a cellulose-based compound, a polyethylene glycol-based compound, and sodium dodecyl sulfate; a 68th combination comprising docetaxel, a polyvinyl pyrrolidone-based compound, a cellulose-based compound, an alginic acid-based compound, and polygamma glutamic acid; a 69th combination comprising docetaxel, a polyvinyl pyrrolidone-based compound, a cellulose-based compound, an alginic acid-based compound, and sodium dodecyl sulfate; a 70th combination comprising docetaxel, a polyvinyl pyrrolidone-based compound, a poloxamer-based compound, a polyethylene glycol-based compound, and an alginic acid-based compound; a 71st combination comprising docetaxel, a polyvinyl pyrrolidone-based compound, a poloxamer-based compound, a polyethylene glycol-based compound, and polygamma glutamic acid; a 72nd combination comprising docetaxel, a polyvinyl pyrrolidone-based compound, a poloxamer-based compound, a polyethylene glycol-based compound, and sodium dodecyl sulfate; a 73rd combination comprising docetaxel, a polyvinyl pyrrolidone-based compound, a polyethylene glycol-based compound, an alginic acid-based compound, and polygamma glutamic acid; a 74th combination comprising docetaxel, a polyvinyl pyrrolidone-based compound, a polyethylene glycol-based compound, an alginic acid-based compound, and sodium dodecyl sulfate; a 75th combination comprising docetaxel, a polyvinyl pyrrolidone-based compound, an alginic acid-based compound, polygamma glutamic acid, and sodium dodecyl sulfate; a 76th combination comprising docetaxel, a cellulose-based compound, a poloxamer-based compound, a polyethylene glycol-based compound, and an alginic acid-based compound; a 77th combination comprising docetaxel, a cellulose-based compound, a poloxamer-based compound, a polyethylene glycol-based compound, and polygamma glutamic acid; a 78th combination comprising docetaxel, a cellulose-based compound, a poloxamer-based compound, a polyethylene glycol-based compound, and sodium dodecyl sulfate; a 79th combination comprising docetaxel, a cellulose-based compound, a polyethylene glycol-based compound, an alginic acid-based compound, and polygamma glutamic acid; an 80th combination comprising docetaxel, a cellulose-based compound, an alginic acid-based compound, polygamma glutamic acid, and sodium dodecyl sulfate; an 81st combination comprising docetaxel, a poloxamer-based compound, a polyethylene glycol-based compound, an alginic acid-based compound, and polygamma glutamic acid; an 82nd combination comprising docetaxel, a poloxamer-based compound, a polyethylene glycol-based compound, an alginic acid-based compound, and sodium dodecyl sulfate; an 83rd combination comprising docetaxel, a poloxamer-based compound, an alginic acid-based compound, polygamma glutamic acid, and sodium dodecyl sulfate; an 84th combination comprising docetaxel, a polyvinyl pyrrolidone-based compound, a cellulose-based compound, a poloxamer-based compound, a polyethylene glycol-based compound, and an alginic acid-based compound; an 85th combination comprising docetaxel, a polyvinyl pyrrolidone-based compound, a cellulose-based compound, a poloxamer-based compound, a polyethylene glycol-based compound, and polygamma glutamic acid; an 86th combination comprising docetaxel, a polyvinyl pyrrolidone-based compound, a cellulose-based compound, a poloxamer-based compound, a polyethylene glycol-based compound, and sodium dodecyl sulfate; an 87th combination comprising docetaxel, a polyvinyl pyrrolidone-based compound, a cellulose-based compound, a poloxamer-based compound, an alginic acid-based compound, and polygamma glutamic acid; an 88th combination comprising docetaxel, a polyvinyl pyrrolidone-based compound, a cellulose-based compound, a poloxamer-based compound, an alginic acid-based compound, and sodium dodecyl sulfate; an 89th combination comprising docetaxel, a polyvinyl pyrrolidone-based compound, a cellulose-based compound, a poloxamer-based compound, polygamma glutamic acid, and sodium dodecyl sulfate; a 90th combination comprising docetaxel, a polyvinyl pyrrolidone-based compound, a cellulose-based compound, a polyethylene glycol-based compound, an alginic acid-based compound, and polygamma glutamic acid; a 91st combination comprising docetaxel, a polyvinyl pyrrolidone-based compound, a cellulose-based compound, a polyethylene glycol-based compound, polygamma glutamic acid, and sodium dodecyl sulfate; a 92nd combination comprising docetaxel, a polyvinyl pyrrolidone-based compound, a cellulose-based compound, an alginic acid-based compound, polygamma glutamic acid, and sodium dodecyl sulfate; a 93rd combination comprising docetaxel, a cellulose-based compound, a poloxamer-based compound, a polyethylene glycol-based compound, an alginic acid-based compound, and polygamma glutamic acid; a 94th combination comprising docetaxel, a cellulose-based compound, a poloxamer-based compound, a polyethylene glycol-based compound, an alginic acid-based compound and sodium dodecyl sulfate; a 95th combination comprising docetaxel, a cellulose-based compound, a polyethylene glycol-based compound, an alginic acid-based compound, polygamma glutamic acid, and sodium dodecyl sulfate; a 96th combination comprising docetaxel, a polyvinyl pyrrolidone-based compound, a cellulose-based compound, a poloxamer-based compound, a polyethylene glycol-based compound, an alginic acid-based compound, and polygamma glutamic acid; a 97th combination comprising docetaxel, a polyvinyl pyrrolidone-based compound, a cellulose-based compound, a poloxamer-based compound, a polyethylene glycol-based compound, an alginic acid-based compound, and sodium dodecyl sulfate; a 98th combination comprising docetaxel, a cellulose-based compound, a poloxamer-based compound, a polyethylene glycol-based compound, an alginic acid-based compound, polygamma glutamic acid, and sodium dodecyl sulfate; a 99th combination comprising docetaxel, a polyvinyl pyrrolidone-based compound, a poloxamer-based compound, a polyethylene glycol-based compound, an alginic acid-based compound, polygamma glutamic acid, and sodium dodecyl sulfate; a 99th combination comprising docetaxel, a polyvinyl pyrrolidone-based compound, a poloxamer-based compound, a cellulose-based compound, an alginic acid-based compound, polygamma glutamic acid, and sodium dodecyl sulfate; a 100th combination comprising docetaxel, a polyvinyl pyrrolidone-based compound, a cellulose-based compound, a poloxamer-based compound, a polyethylene glycol-based compound, polygamma glutamic acid, and sodium dodecyl sulfate; a 101st combination comprising docetaxel, a polyvinyl pyrrolidone-based compound, a cellulose-based compound, a poloxamer-based compound, a polyethylene glycol-based compound, an alginic acid-based compound, and sodium dodecyl sulfate; a 102nd combination comprising docetaxel, a polyvinyl pyrrolidone-based compound, a cellulose-based compound, a poloxamer-based compound, a polyethylene glycol-based compound, an alginic acid-based compound, and polygamma glutamic acid; and a 103rd combination comprising docetaxel, a polyvinyl pyrrolidone-based compound, a cellulose-based compound, a poloxamer-based compound, a polyethylene glycol-based compound, an alginic acid-based compound, polygamma glutamic acid, and sodium dodecyl sulfate.

The pharmaceutical composition of the present invention may further contain one or more selected from among magnesium oxide (MgO), bentonite mineral, hydrotalcite, and magnesium hydroxide in the 1st to 103rd combinations. When magnesium oxide or bentonite mineral are further included, the blood solubility of docetaxel can be improved, and thus the pharmaceutical composition can have substantially excellent bioavailability. In this regard, further containing magnesium oxide or bentonite mineral is preferable.

The pharmaceutical composition of the present invention may further contain one or more materials selected from the group consisting of a crystallization inhibitor, a swellable polymer, an enteric coating, a foam generator, and a swellable excipient.

Specifically, in the present invention, the crystallization inhibitor may indicate a polymer capable of delaying the return of a compound, such as docetaxel, from a dissolved state in a solvent to a crystalline form so as to maintain an amorphous or non-crystalline form of each compound. Loading the drug into the crystallization inhibitor delays the crystallization time of the drug. Therefore, the solubility of the drug in the body enhances, and the blood concentration of the drug increases. Any crystallization inhibitor available typically can be used without limitation, but specifically, the crystallization inhibitor may be a polyoxyethylene sorbitan fatty acid ester compound, a lecithin compound, a fatty acid compound, a glycerol fatty acid ester compound, a sorbitan fatty acid ester compound, oils, sodium dodecyl sulfate, sodium stearyl fumarate, stearyl acid, lauric acid, and carrageenan.

In addition, in the case of the polyoxyethylene sorbitan fatty acid ester compound, commercially available Tween-based surfactants are the most representative and take the form of an ester bond between a fatty acid and ethylene oxide. More specifically, the polyoxyethylene sorbitan fatty acid ester compound may be polyoxyethylene sorbitan monolaurate (Tween 20), polyoxyethylene sorbitan monopalmitate (Tween 40), polyoxyethylene glycol sorbitan monostearate (Tween 60), Tween65, polyoxyethylene sorbitan monooleate (Tween80), polyoxyethylene sorbitan trioleate (Tween85), etc.

In addition, the lecithin compound is a material for lecithin and its derivatives and may be phospholipids, phosphatidyl choline, mixed phospholipids, sodium cholate, hydroxylated phospholipids, hydroxylated lecithin, and the like.

In addition, the fatty acid compound may be butyric acid, caproic acid, caprylic acid, capric acid, stearic acid, lauric acid, oleic acid, myristoleic acid, palmitoleic acid, oleic acid, linoleic acid, α-linolenic acid, γ-linolenic acid, gadoleic acid, eicosadienoic acid, eicosapentanoic acid, arachidonic acid, erucic acid, docosadienoic acid, docosatrienoic acid, docosapentaenoic acid, docosahexaenoic acid, adrenic acid, nervonic acid, and the like.

In addition, the glycerol fatty acid esters may be polyglycerol fatty acid esters, polyglycerol polyricinoleate, polyoxyethyleneglycerol triricinoleate, cremophor EL etc.

In addition, the sorbitan fatty acid esters may be sorbitan monolaurate (Span 20), sorbitan monooleate (Span 80), etc.

In addition, the oils may be soybean oil, MCT (medium-chain triglyceride) oil, castor oil, and the like. When the above-mentioned crystallization inhibitor is further included, the solubility and dispersibility of a poorly soluble drug can be improved. In this regard, further containing the crystallization inhibitor is preferable.

In the present invention, the swellable polymer is a material that can help control the drug release rate in formulating a drug, and any commonly used swellable polymer may be used without limitation. Specifically, the swellable polymer may be carboxymethyl cellulose calcium, alginate, sodium carboxymethyl cellulose, methyl cellulose, ethyl cellulose, polyethylene oxide, locust bean gum, guar gum, xanthan gum, acacia gum, tragacanth gum, agar, gelatin, polymethylmethacrylate, carbomer, polycarbophil, polyvinyl acetate, polyvinylpyrrolidone-polyvinyl acrylate copolymer, polyvinyl alcohol-polyethylene glycol copolymer, polyvinylpyrrolidone-polyvinyl acetate copolymer, bentonite, hectorite, carrageenan, ceratonia, cetostearyl alcohol, hydroxypropyl starch, magnesium aluminum silicate, polydextrose, poly(methylvinyl ether/maleic anhydrous), propylene glycol alginate and saponite, and the like.

In the present invention, the enteric coating means a material that inhibits recrystallization of the drug by gastric fluid and increases the absorption rate, so that absorption of the drug is not hindered due to recrystallization by gastric fluid in the stomach. The enteric coating can be used to select the site within the intestine where the drug is released. In particular, in the case of docetaxel, the bioavailability varies over time depending on the pH of each site in the intestine, and thus a more optimized dosage form can be selected using this feature. In this regard, it may be preferable to use an enteric coating. A commonly used swellable polymer may be used without limitation. Specifically, the enteric coating may be hydroxypropyl-methyl cellulose phthalate, zein, shellac, Eudragit, and the like.

In the present invention, the foam generator is a material capable of imparting floating properties to a tableted drug and is characterized in that it is selected from the group consisting of sodium carbonate, sodium bicarbonate, potassium carbonate, potassium bicarbonate, and citric acid.

In addition, in the present invention, a commonly used swellable polymer may be used without limitation as the swellable excipient. Specifically, the swellable excipient may be chitosan, xanthan gum, carboxymethyl cellulose, natural cellulose, pectin, hyaluronic acid, polyacrylate, polyethylene oxide, polypropylene oxide, monosaccharides, methacrylic acid-ethyl acrylate copolymers, shellacs, carbopols (carbomer, carboxyvinyl polymer), polyvinyl alcohol, hydroxypropylmethylcellulose phthalate-based compounds, cellulose acetate phthalate, cellulose acetate succinate, acetate succinate, hydroxypropylmethylcellulose hydroxypropylmethylacetate succinate, carboxymethylcellulose, carboxymethylethyl cellulose, cellulose acetate phthalate compounds, hydroxypropyl cellulose compounds, ethyl cellulose compounds, methyl cellulose compounds, polyvinyl acetate phthalate, silicon dioxide, calcium silicate, milk sugar, starch, lactose, mannitol, kaolin inorganic salt, powdered sugar, powdered cellulose derivatives, microcrystalline cellulose, and the like.

When the composition of the present invention was formulated in the form of a film coating, a semi-permeable membrane coating, a water-insoluble coating, a tablet, a double tablet, a gastric retention tablet, etc. by using the above coating agent, it was confirmed that the blood concentration of the drug was very high. Thus, the formation of the coating agents as described above may help improve the solubility of the drug, etc.

In the present invention, the docetaxel or a pharmaceutically acceptable salt thereof and other polymeric compounds, that is, one or more of a cellulose-based compound, a poloxamer-based compound, a polyethylene glycol-based compound, and a polyvinyl pyrrolidone-based compound, may satisfy the weight ratio range of 1:1 to 1:12. If the above range is not satisfied, the solubility of docetaxel, a poorly soluble drug, cannot be sufficiently increased. Or, if the weight ratio of one or more of a cellulose-based compound, a poloxamer-based compound, a polyethylene glycol-based compound, and a polyvinyl pyrrolidone-based compound exceeds 12, the content of the drug can be lowered, and the possibility of commercialization can be lowered, and the release of the drug can be delayed, which may cause a problem that absorption in the body and digestive behavior are inefficient.

The pharmaceutical composition of the present invention may further include a pharmaceutically acceptable carrier and may be formulated for oral or parenteral human or veterinary use. When formulating the pharmaceutical composition of the present invention, diluents or excipients, such as fillers, bulking agents, binders, wetting agents, disintegrants, surfactants, or the like may be used in addition to the components listed above. Solid formulations for oral administration include tablets, pills, powders, granules, capsules, etc. These solid formulations may be prepared by mixing the pharmaceutical composition containing the compound of the present invention with at least one excipient, for example, starch, calcium carbonate, sucrose, lactose, gelatin, mannitol, etc. Furthermore, in addition to simple excipients, lubricants, such as magnesium stearate and talc, may be used. Liquid formulations for oral use include a suspension, a liquid for internal use, an emulsion, syrup, and the like. In addition to water and liquid paraffin, which are commonly used simple diluents, various excipients, for example, a wetting agent, a sweetener, a flavoring agent, a preserving agent, or the like may be added. Formulations for parenteral administration include a sterilized aqueous solution, a non-aqueous solvent, a suspension, an emulsion, a lyophilized formulation, and a suppository formulation. As the non-aqueous solvent or the suspension, propylene glycol, polyethylene glycol, a plant oil such as olive oil, an injectable ester such as ethyloleate, or the like may be used. As a base for suppositories, witepsol, macrogol, tween 61, cacao butter, laurin butter, glycerogelatin, or the like may be used.

The pharmaceutical composition of the present invention may be administered orally or parenterally, depending on the desired method. In the case of parenteral administration, it is preferable to select a topical application or injection methods, such as intraperitoneal injection, intrarectal injection, subcutaneous injection, intravenous injection, intramuscular injection, or intrathoracic injection.

Also, the pharmaceutical composition according to the present invention may be administered by inhalation. It can deliver drugs directly to the lungs without causing toxicity and exhibit longer duration of action at lower doses. Administration for inhalation may use a pharmaceutical formulation that includes respirable particles or droplets containing the drug and thus can be inhaled through the respiratory tract, nasal cavity, etc. For such administration for inhalation, for example, but without limitation, either a dry powder inhaler device (DPI) or a pressurized metered dose inhaler (pMDI) may be used. The drug particles are lightly compacted into a frangible matrix which is contained, for example, within the delivery device (a dry powder inhaler). Upon actuation, the delivery device abrades a portion of the drug particles from the matrix and disperses them in the inspiratory breath delivering the drug particles to the respiratory tract. Alternatively, the drug particles may be a free flowing powder contained within a reservoir in the delivery device (a dry powder inhaler). The reservoir can be an integral chamber within the device, a capsule, a blister, or a reservoir of similar performance that is inserted into the device prior to actuation. Upon actuation, the device disperses a portion of the drug particles from the reservoir and disperses them in the inhalation breath delivering the drug particles to the respiratory tract.

The composition of the present invention is administered in a pharmaceutically effective amount. As used herein, the term pharmaceutically effective amount means an amount enough to treat a disease at a reasonable benefit/risk ratio applicable to medical treatment, and a level of effective dosage may be determined according to factors including a patient's weight, gender, age, health condition, severity, activity of a drug, sensitivity to a drug, an administration time, an administration route and excretion rate, a treatment period and a concurrently used drug, as well as other factors well known in a medical field. The pharmaceutical composition of the present invention may be administered as an individual therapeutic agent or in combination with other therapeutic agents, and may be administered sequentially or simultaneously with conventional therapeutic agents. Single dose or multiple doses may be administered. Considering all of the above factors, it is important to administer an amount that can bring about the maximum effect with a minimal amount without side effects. The amount can be easily determined by a person skilled in the art.

For example, the pharmaceutical composition, according to the present invention, may be administered at a dose of 0.0001 to 500 mg/kg, preferably 0.001 to 500 mg/kg. The administration may be performed once a day to 5 times a day. In addition, the administration may be performed once every 2 days to once every 10 days depending on the purpose.

The pharmaceutical composition of the present invention may contain 0.1 to 50 wt % of docetaxel or a pharmaceutically acceptable salt thereof and 0.2 to 90 wt % of one or more of a cellulose-based compound, a poloxamer-based compound, a polyethylene glycol-based compound, and a polyvinyl pyrrolidone-based compound, based on the total weight of the composition being 100 wt %. The composition may further include 0.1 to 70 wt % of one or more of magnesium oxide (MgO), bentonite mineral, hydrotalcite, and magnesium hydroxide, and may further include 1 to 30 wt % of one or more materials selected from the group consisting of a crystallization inhibitor, a swellable polymer, an enteric coating, a foam generator, and a swellable excipient.

The pharmaceutical composition of the present invention may be for anti-inflammatory, antiviral, and/or anticancer purposes.

In the present invention, anti-inflammatory means having an effect capable of alleviating inflammatory reactions caused by infectious, traumatic, endogenous, inflammatory, degenerative or autoimmune causes.

In the present invention, antiviral means having an antiviral effect, and having a virus reproduction-inhibitory effect and an antibiotic function against malaria infection causing a viral disease or Epstein Barr Virus (EBV), hepatitis B virus, hepatitis C virus, HIV, HTLV 1, Varicella-Zoster Virus (VZV), Human Papilloma Virus (HPV), Corona virus, such as SARS-CoV and/or SARS-CoV2, rhinovirus, adenovirus, RS virus, parainfluenza virus, which cause a cold or respiratory illness, an infection caused by RS virus or the like, and other viruses such as retroviruses.

In addition, the meaning of anticancer in the present invention means having anticancer activity mainly by acting directly on DNA to block DNA replication, transcription, and translation processes or by interfering with the synthesis of nucleic acid precursors in the metabolic pathway and by inhibiting cell division. Specifically, anticancer means having a therapeutic effect on cancer and carcinomas arising from breast, prostate, kidney, bladder, or colon tissues, including fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma; and adipocyte tumors, such as lipoma, fibrolipoma, lipoblastoma, lipomatosis, hibemoma, hemangioma, and/or neoplastic diseases appearing in adipose tissue, such as liposarcoma. More specifically, the cancer may be breast cancer, biliary tract cancer, gallbladder cancer, pancreatic cancer, colon cancer, uterine cancer, esophageal cancer, stomach cancer, brain cancer, rectal cancer, lung cancer, bladder cancer, kidney cancer, ovarian cancer, prostate cancer, uterine cancer, head and neck cancer, skin cancer, blood cancer, and liver cancer.

The composition of the present invention may additionally include ingredients that do not increase the efficacy of the drug, but can be commonly used in pharmaceutical compositions to improve odor, taste, visual appearance, and the like. In addition, the pharmaceutical composition of the present invention may additionally contain pharmaceutically acceptable additives. Pharmaceutically acceptable additives include, for example, but without limitation, starch, gelatinized starch, microcrystalline cellulose, milk sugar, povidone, colloidal silicon dioxide, calcium hydrogen phosphate, lactose, mannitol, crude maltose, gum arabic, pregelatinized starch, corn starch, powdered cellulose, hydroxypropyl sodium starch glycolate, carnauba wax, synthetic aluminum silicate, stearic acid, magnesium stearate, aluminum stearate, calcium stearate, white sugar, dextrose, sorbitol, and talc.

The present invention provides a method for preparing a pharmaceutical composition, the method comprising the steps of: preparing a first dissolved product by dissolving one or more compounds selected from among a polyvinyl pyrrolidone-based compound, a cellulose-based compound, a poloxamer-based compound, and a polyethylene glycol-based compound in an organic solvent; and preparing a second dissolved product by adding and stirring docetaxel or a pharmaceutically acceptable salt thereof to the first dissolved product. In the above, a method of first dissolving one or more compounds selected from among a polyvinyl pyrrolidone-based compound, a cellulose-based compound, a poloxamer-based compound, and a polyethylene glycol-based compound, which correspond to a polymer compound, is exemplified. However, a first dissolved product may be prepared by first dissolving docetaxel or a pharmaceutically acceptable salt thereof, and then a second dissolved product may be prepared by dissolving the one or more compounds selected from among a polyvinyl pyrrolidone-based compound, a cellulose-based compound, a poloxamer-based compound, and a polyethylene glycol-based compound in the first dissolved product. Changing or substituting the mixing order thereof is obvious to a person skilled in the art and falls within the scope of the present invention.

The organic solvent may be one or more selected from among ethanol, methanol, propanol, butanol, and acetonitrile. However, an anhydrous organic solvent may be more preferable when it comes to improving reactivity, and anhydrous ethanol is the most preferable.

In the preparation method of the present invention, if necessary, water may be added to the organic solvent, or water may be used.

In addition, the method for preparing the pharmaceutical composition may further include the steps of: preparing powders by evaporating the second dissolved product; and/or mixing one or more compounds selected from among a cellulose-based compound, a poloxamer-based compound, a polyethylene glycol-based compound, polygamma glutamic acid, and sodium dodecyl sulfate in a powdered form with the powders and milling and/or grinding the mixture.

In addition, the method for preparing the pharmaceutical composition may further include the steps of: preparing powders by evaporating the second dissolved product; and/or physically mixing one or more of magnesium oxide (MgO), bentonite mineral, hydrotalcite, and magnesium hydroxide with the powders.

In addition, the method for preparing the pharmaceutical composition may further include the steps of: preparing a third dissolved product by dispersing one or more of magnesium oxide (MgO), bentonite mineral, hydrotalcite, and magnesium hydroxide in the second dissolved product; and drying the third dissolved product.

Physical mixing in the present invention refers to a method of physically mixing solid materials, and milling or grinding methods may be used. In addition, when one or more selected from among magnesium oxide (MgO), bentonite mineral, hydrotalcite, and magnesium hydroxide are mixed in the above step, the solubility of the poorly soluble drug can be significantly improved, and thus the desired effect of increasing the bioavailability can be obtained. In this regard, mixing one or more selected from among magnesium oxide (MgO), bentonite mineral, hydrotalcite, and magnesium hydroxide in the above step is preferable.

In the present invention, the drying step may be, without limitation, any method for evaporating the solvent, but spray drying may be the most preferable.

In addition, the step for evaporating the solvent in the preparation method of the present invention is characterized by using a solvent evaporation method other than a hot melting method, which is generally used in the preparation of a solid dispersion. The hot melting method uses a screw, which is an expensive piece of equipment, and has disadvantages, such as the temperature control that may occur in the process, the phenomenon of fine particle grinding of the device, and decreased effect of a drug having poor stability due to sensitivity to the temperature. Thus, the hot melting method is not suitable for formulation of poorly soluble drugs. Therefore, the present invention improves the ease of preparation through a rotary evaporator or a spray dryer, using a commonly used ethanol solvent, through a solvent evaporation method.

Hereinafter, a more specific method of implementing the present invention will be described in detail, but the present invention is not limited thereto.

### EXAMPLES 1 TO 10: PREPARATION OF COMPOSITIONS OF THE PRESENT INVENTION

### Example 1

In a round bottom flask, 2 g of docetaxel is added to a solution of 2 g of PEG400 dissolved in 100 mL of anhydrous ethanol. After stirring the solution for about 1 hour, the solvent is removed from the solution thoroughly through a rotary evaporator to obtain white powders.

### Example 2

In a round bottom flask, 2 g of docetaxel is added to a solution of 4 g of PEG1400 dissolved in 100 mL of anhydrous ethanol. After stirring the solution for about 1 hour, the solvent is removed from the solution thoroughly through a rotary evaporator to obtain white powders.

### Example 3

In a round bottom flask, 2 g of docetaxel is added to a solution of 2 g of poloxamer 407 dissolved in 100 mL of anhydrous ethanol. After stirring the solution for about 1 hour, the solvent is removed from the solution thoroughly through a rotary evaporator to obtain white powders.

### Example 4

In a round bottom flask, 2 g of docetaxel is added to a solution of 2 g of PVP (polyvinylpyrrolidone K12 (MW 11,000-12,000)) dissolved in 100 mL of anhydrous ethanol. After stirring the solution for about 1 hour, the solvent is removed from the solution thoroughly through a rotary evaporator to obtain white powders.

### Example 5

In a round bottom flask, 1 g of docetaxel is added to a solution of 0.5 g of HPMC (6 mPas) dissolved in 100 mL of anhydrous ethanol. After stirring the solution for about 1 hour, the solvent is removed from the solution thoroughly through a rotary evaporator to obtain white powders.

### Example 6

In a round bottom flask, 2 g of docetaxel is added to a solution of 2 g of PEG400 dissolved in 100 mL of anhydrous ethanol. After stirring the solution for about 1 hour, the solvent is removed from the solution thoroughly through a rotary evaporator, and then powders are obtained. After adding 1 g of MgO to 0.5 g of the powders and grinding the mixture, white powders are obtained.

### Example 7

In a round bottom flask, 1 g of docetaxel is added to a solution of 2 g of PEG1400 dissolved in 100 mL of anhydrous ethanol. After stirring the solution for about 1 hour, the solvent is removed from the solution thoroughly through a rotary evaporator, and then 3 g of powders are obtained. After adding 2 g of MgO to 3 g of the powders and grinding the mixture, white powders are obtained.

### Example 8

In a round bottom flask, 1 g of docetaxel is added to a solution of 1 g of poloxamer 407 dissolved in 100 mL of anhydrous ethanol. After stirring the solution for about 1 hour, the solvent was removed from the solution thoroughly through a rotary evaporator to prepare powders. After adding 2 g of MgO to 2 g of the powders and grinding the mixture, white powders are obtained.

### Example 9

In a round bottom flask, 1 g of docetaxel is added to a solution of 1 g of PVP (polyvinylpyrrolidone K12 (MW 11,000-12,000)) dissolved in 100 mL of anhydrous ethanol. After stirring the solution for about 1 hour, the solvent is removed from the solution thoroughly through a rotary evaporator, and then powders are obtained. After adding 2 g of MgO to 2 g of the powders and grinding the mixture, white powders are obtained.

### Example 10

In a round bottom flask, 1 g of docetaxel is added to a solution of 0.5 g of HPMC (6 mPas) dissolved in 100 mL of anhydrous ethanol. After stirring the solution for about 1 hour, the solvent is removed from the solution thoroughly through a rotary evaporator, and then powders are obtained. After adding 1 g of MgO to 1.5 g of the powders and grinding the mixture, white powders are obtained.

### Example 11

In a round bottom flask, 1 g of docetaxel is added to a solution of 0.4 g of PVP (polyvinylpyrrolidone K12 (MW 11,000-12,000)) dissolved in 10 mL of anhydrous ethanol. After stirring the solution for about 1 hour, the solvent is removed from the solution thoroughly through a rotary evaporator, and then powders are obtained. After adding 2 g of MgO to 1.4 g of the powders and grinding the mixture, white powders are obtained.

### Example 12

0.16 g of alginate, 1 g of docetaxel, and 2 g of MgO are mixed and ground to obtain white powders.

### Example 13

In a round bottom flask, 1 g of docetaxel is added to a solution of 0.4 g of PVP (polyvinylpyrrolidone K12 (MW 11,000-12,000)) dissolved in 10 mL of anhydrous ethanol. After stirring the solution for about 1 hour, the solvent is removed from the solution thoroughly through a rotary evaporator, and then powders are obtained. After adding 0.16 g of alginate and 2 g of MgO to 1.4 g of the powders and grinding the mixture, white powders are obtained.

### Example 14

In a round bottom flask, 1 g of docetaxel is added to a solution of 0.4 g of PVP (polyvinylpyrrolidone K12 (MW 11,000-12,000)) dissolved in 10 mL of anhydrous ethanol. After stirring the solution for about 1 hour, the solvent is removed from the solution thoroughly through a rotary evaporator, and then powders are obtained. After adding 0.155 g of MMT to the powders and grinding the mixture, white powders are obtained.

### Example 15

In a round bottom flask, 1 g of DTX is dissolved in 10 mL of anhydrous ethanol to make a solution. After dissolving 0.06 g of alginate and 0.12 g of MMT in distilled water, the solvent is removed from the solution thoroughly through a rotary evaporator to obtain powders. After adding the alginate-MMT powders to the DTX solution and stirring for about 1 hour, the solvent is removed from the solution thoroughly through a rotary evaporator to obtain white powders.

### Example 16

In a round bottom flask, a solution is made by dissolving 1 g of docetaxel in a solution of 0.4 g of PVP (polyvinylpyrrolidone K12 (MW 11,000-12,000)) dissolved in 10 mL of anhydrous ethanol. After dissolving 0.08 g of alginate and 0.165 g of MMT in distilled water, the solvent is removed from the solution thoroughly through a rotary evaporator to obtain powders. After adding the alginate-MMT powders to the DTX-PVP solution and stirring for about 1 hour, the solvent is removed from the solution thoroughly through a rotary evaporator to obtain white powders.

### Example 17

In a round bottom flask, a solution is made by dissolving 1 g of docetaxel in a solution of 0.4 g of PVP (polyvinylpyrrolidone K12 (MW 11,000-12,000)) dissolved in 10 mL of anhydrous ethanol. After adding 0.015 g of MMT to the DTX-PVP solution and stirring for about 1 hour, the solvent is removed from the solution thoroughly through a rotary evaporator to obtain white powders.

### Example 18

In a round bottom flask, a solution is made by dissolving 1 g of docetaxel in a solution of 0.4 g of PVP (polyvinylpyrrolidone K12 (MW 11,000-12,000)) dissolved in 10 mL of anhydrous ethanol. After adding 0.075 g of MMT to the DTX-PVP solution and stirring for about 1 hour, the solvent is removed from the solution thoroughly through a rotary evaporator to obtain white powders.

### Example 19

In a round bottom flask, a solution is made by dissolving 1 g of docetaxel in a solution of 0.4 g of PVP (polyvinylpyrrolidone K12 (MW 11,000-12,000)) dissolved in 10 mL of anhydrous ethanol. After adding 0.35 g of MMT to the DTX-PVP solution and stirring for about 1 hour, the solvent is removed from the solution thoroughly through a rotary evaporator to obtain white powders.

### Example 20

In a round bottom flask, a solution (DTX-PVP solution) is made by dissolving 1 g of docetaxel in a solution of 0.4 g of PVP (polyvinylpyrrolidone K12 (MW 11,000-12,000)) dissolved in 10 mL of anhydrous ethanol. Subsequently, after dissolving 0.08 g of alginate and 0.15 g of MMT in distilled water, the solvent is removed from the solution thoroughly through a rotary evaporator to obtain 0.23 g of alginate-MMT powders. After adding 0.23 g of the alginate-MMT powders to the DTX-PVP solution and stirring for about 1 hour, the solvent is removed from the solution thoroughly through a rotary evaporator to obtain white powders.

### Example 21

In a round bottom flask, a solution (DTX-PVP solution) is made by dissolving 1 g of docetaxel in a solution of 0.4 g of PVP (polyvinylpyrrolidone K12 (MW 11,000-12,000)) dissolved in 10 mL of anhydrous ethanol. After dissolving 0.08 g of alginate and 0.08 g of MMT in distilled water, the solvent is removed from the solution thoroughly through a rotary evaporator to obtain 0.16 g of alginate-MMT powders. After adding 0.16 g of the alginate-MMT powders to the DTX-PVP solution and stirring for about 1 hour, the solvent is removed from the solution thoroughly through a rotary evaporator to obtain white powders.

### Example 22

In a round bottom flask, a solution (DTX-PVP solution) is made by dissolving 1 g of docetaxel in a solution of 0.4 g of PVP (polyvinylpyrrolidone) dissolved in 10 mL of anhydrous ethanol. After dissolving 0.1 g of alginate and 0.375 g of MMT in distilled water, the solvent is removed from the solution thoroughly through a rotary evaporator to obtain 0.475 g of alginate-MMT powders. After adding 0.475 g of the alginate-MMT powders to the DTX-PVP solution and stirring for about 1 hour, the solvent is removed from the solution thoroughly through a rotary evaporator to obtain white powders.

### Example 23

In a round bottom flask, 1 g of docetaxel is added to a solution of 4 g of PVP dissolved in 50 mL of anhydrous ethanol. After stirring the solution for about 1 hour, the solvent is removed from the solution thoroughly through a rotary evaporator to obtain white powders.

### Example 24

In a round bottom flask, 1 g of docetaxel is added to a solution of 8 g of PVP dissolved in 50 mL of anhydrous ethanol. After stirring the solution for about 1 hour, the solvent is removed from the solution thoroughly through a rotary evaporator to obtain white powders.

### Example 25

In a round bottom flask, 1 g of docetaxel is added to a solution of 2 g of PVP dissolved in 50 mL of anhydrous ethanol. After stirring the solution for about 1 hour, the solvent is removed from the solution thoroughly through a rotary evaporator, and then 3 g of docetaxel-PVP powders are obtained. After adding 2 g of MgO to 3 g of the docetaxel-PVP powders and mixing well, white powders are obtained.

### Example 26

In a round bottom flask, 1 g of docetaxel is added to a solution of 4 g of PVP dissolved in 50 mL of anhydrous ethanol. After stirring the solution for about 1 hour, the solvent is removed from the solution thoroughly through a rotary evaporator, and then 5 g of docetaxel-PVP powders are obtained. After adding 2 g of MgO to 5 g of the docetaxel-PVP powders and mixing well, white powders are obtained.

### Example 27

In a round bottom flask, 1 g of docetaxel is added to a solution of 8 g of PVP (polyvinylpyrrolidone K12 (MW 11,000-12,000)) dissolved in 50 mL of anhydrous ethanol. After stirring the solution for about 1 hour, the solvent is removed from the solution thoroughly through a rotary evaporator, and then 9 g of docetaxel-PVP powders are obtained. After adding 2 g of MgO to 9 g of the docetaxel-PVP powders and mixing well, white powders are obtained.

### Example 28

In a round bottom flask, 1 g of docetaxel is added to a solution of 4 g of PVP (polyvinylpyrrolidone K12 (MW 11,000-12,000)) dissolved in 50 mL of anhydrous ethanol. After stirring the solution for about 1 hour, 0.8 g of HPMC (6 mPas) is added, the solution is stirred for about 1 hour, the solvent is removed from the solution thoroughly through a rotary evaporator, and then 5.8 g of docetaxel-PVP-HPMC powders are obtained. After adding 2 g of MgO to 5.8 g of the docetaxel-PVP-HPMC powders and mixing well, white powders are obtained.

### Example 29

In a round bottom flask, 1 g of docetaxel is added to a solution of 4 g of PVP (polyvinylpyrrolidone K12 (MW 11,000-12,000)) dissolved in 50 mL of anhydrous ethanol. After stirring the solution for about 1 hour, 0.8 g of poloxamer 407 is added, the solution is stirred for about 1 hour, the solvent is removed from the solution thoroughly through a rotary evaporator, and then 5.8 g of docetaxel-PVP-poloxamer powders are obtained. After adding 2 g of MgO to 5.8 g of the docetaxel-PVP-poloxamer powders and mixing well, white powders are obtained.

### Example 30

In a round bottom flask, 1 g of docetaxel is added to a solution of 4 g of PVP (polyvinylpyrrolidone K12 (MW 11,000-12,000)) dissolved in 50 mL of anhydrous ethanol. After stirring the solution for about 1 hour, 0.9 g of HPMC (6 mPas) and 0.9 g of poloxamer 407 are added, the solution is stirred for about 1 hour, the solvent is removed from the solution thoroughly through a rotary evaporator, and then 6.8 g of docetaxel-PVP-HPMC powders are obtained. After adding 2 g of MgO to 6.8 g of the docetaxel-PVP-HPMC powders and mixing well, white powders are obtained.

### Example 31

In a round bottom flask, 1 g of docetaxel is added to a solution of 4 g of PVP (polyvinylpyrrolidone K12 (MW 11,000-12,000)) dissolved in 50 mL of anhydrous ethanol. After stirring the solution for about 1 hour, the solvent is removed from the solution thoroughly through a rotary evaporator, and then 5 g of docetaxel-PVP powders are obtained.

After adding 2 g of MgO and 0.37 g of Na-alginate to 5 g of the docetaxel-PVP powders and mixing well, white powders are obtained.

### Example 32

In a round bottom flask, 1 g of docetaxel is added to a solution of 4 g of PVP (polyvinylpyrrolidone K12 (MW 11,000-12,000)) dissolved in 50 mL of anhydrous ethanol, and the solution is stirred for about 1 hour. After adding 2 g of MgO and stirring for about 1 hour, the solvent is removed from the solution thoroughly through a rotary evaporator to obtain white powders.

### Example 33

In a round bottom flask, 1 g of docetaxel is added to a solution of 4 g of PVP (polyvinylpyrrolidone K12 (MW 11,000-12,000)) dissolved in 50 mL of anhydrous ethanol, and the solution is stirred for about 1 hour. After adding 2 g of MgO and stirring for about 1 hour, the solvent is removed from the solution thoroughly through a rotary evaporator to obtain white powders.

### Examples 34 to 43

Examples 34 to 43 were prepared in the same manner as in Example 33, except that each component was included in the ratio shown in Table 1 below.

TABLE 1

| | | | | poloxamer | PVP | | | | unit |
|---|---|---|---|---|---|---|---|---|---|
| Classification | DTX | PEG400 | PEG1400 | 407 | K30 | HPMC 6 | MgO | total | (weight) |
| Example 34 | 50 | 50 | 0 | 0 | 0 | 0 | 0 | 100 | wt % |
| | 1 | 1 | 0 | 0 | 0 | 0 | 0 | 2 | ratio |
| Example 35 | 33.33 | 0 | 66.67 | 0 | 0 | 0 | 0 | 100 | wt % |
| | 1 | 0 | 2 | 0 | 0 | 0 | 0 | 3 | ratio |
| Example 36 | 50 | 0 | 0 | 50 | 0 | 0 | 0 | 100 | wt % |
| | 1 | 0 | 0 | 1 | 0 | 0 | 0 | 2 | ratio |
| Example 37 | 50 | 0 | 0 | 0 | 50 | 0 | 0 | 100 | wt % |
| | 1 | 0 | 0 | 0 | 1 | 0 | 0 | 2 | ratio |
| Example 38 | 66.67 | 0 | 0 | 0 | 0 | 33.33 | 0 | 100 | wt % |
| | 1 | 0 | 0 | 0 | 0 | 0.5 | 0 | 1.5 | ratio |
| Example 39 | 25 | 25 | 0 | 0 | 0 | 0 | 50 | 100 | wt % |
| | 1 | 1 | 0 | 0 | 0 | 0 | 2 | 4 | ratio |
| Example 40 | 20 | 0 | 40 | 0 | 0 | 0 | 40 | 100 | wt % |
| | 1 | 0 | 2 | 0 | 0 | 0 | 2 | 5 | ratio |
| Example 41 | 25 | 0 | 0 | 25 | 0 | 0 | 50 | 100 | wt % |
| | 1 | 0 | 0 | 1 | 0 | 0 | 2 | 4 | ratio |
| Example 42 | 25 | 0 | 0 | 0 | 25 | 0 | 50 | 100 | wt % |
| | 1 | 0 | 0 | 0 | 1 | 0 | 2 | 4 | ratio |
| Example 43 | 40 | 0 | 0 | 0 | 0 | 20 | 40 | 100 | wt % |
| | 1 | 0 | 0 | 0 | 0 | 0.5 | 2 | 3.5 | ratio |

### Examples 44 to 54

Examples 44 to 54 were prepared in the same manner as in Example 33, except that each component was included in the ratio shown in Table 2 below.

TABLE 2

| Classi- | | PVP | | | | | unit |
|---|---|---|---|---|---|---|---|
| fication | DTX | K25 | alginate | MMT | MgO | total | (weight) |
| Example | 29.41 | 11.76 | 0 | 0 | 58.83 | 100 | wt % |
| 44 | 1 | 0.4 | 0 | 0 | 2 | 3.4 | ratio |
| Example | 31.65 | 0 | 5.06 | 0 | 63.29 | 100 | wt % |
| 45 | 1 | 0 | 0.16 | 0 | 2 | 3.16 | ratio |
| Example | 28.09 | 11.24 | 4.49 | 0 | 56.18 | 100 | wt % |
| 46 | 1 | 0.4 | 0.16 | 0 | 2 | 3.56 | ratio |
| Example | 64.31 | 25.72 | 0 | 9.97 | 0 | 100 | wt % |
| 47 | 1 | 0.4 | 0 | 0.155 | 0 | 1.555 | ratio |
| Example | 84.75 | 0 | 5.08 | 10.17 | 0 | 100 | wt % |
| 48 | 1 | 0 | 0.06 | 0.12 | 0 | 1.18 | ratio |
| Example | 60.79 | 24.32 | 4.86 | 10.03 | 0 | 100 | wt % |
| 49 | 1 | 0.4 | 0.08 | 0.165 | 0 | 1.645 | ratio |
| Example | 70.67 | 28.27 | 0 | 1.06 | 0 | 100 | wt % |
| 50 | 1 | 0.4 | 0 | 0.015 | 0 | 1.415 | ratio |
| Example | 67.8 | 27.12 | 0 | 5.08 | 0 | 100 | wt % |
| 51 | 1 | 0.4 | 0 | 0.075 | 0 | 1.475 | ratio |
| Example | 57.14 | 22.86 | 0 | 20 | 0 | 100 | wt % |
| 52 | 1 | 0.4 | 0 | 0.35 | 0 | 1.75 | ratio |
| Example | 66.89 | 26.76 | 5.35 | 1 | 0 | 100 | wt % |
| 53 | 1 | 0.4 | 0.08 | 0.015 | 0 | 1.495 | ratio |
| Example | 64.1 | 25.64 | 5.13 | 5.13 | 0 | 100 | wt % |
| 54 | 1 | 0.4 | 0.08 | 0.08 | 0 | 1.56 | ratio |
| Example | 53.33 | 21.34 | 5.33 | 20 | 0 | 100 | wt % |
| 55 | 1 | 0.4 | 0.1 | 0.375 | 0 | 1.875 | ratio |

### Examples 56 to 67

Examples 56 to 67 were prepared in the same manner as in Example 33, except that each component was included in the ratio shown in Table 3 below.

TABLE 3

| | | PVP | | | poloxamer | Na- | | unit |
|---|---|---|---|---|---|---|---|---|
| | DTX | K60 | HPMC6 | MgO | 407 | alginate | total | (weight) |
| Example | 25 | 25 | 0 | 50 | 0 | 0 | 100 | wt % |
| 56 | 1 | 1 | 0 | 2 | 0 | 0 | 4 | ratio |
| Example | 20 | 80 | 0 | 0 | 0 | 0 | 100 | wt % |
| 57 | 1 | 4 | 0 | 0 | 0 | 0 | 5 | ratio |
| Example | 11.11 | 88.89 | 0 | 0 | 0 | 0 | 100 | wt % |
| 58 | 1 | 8 | 0 | 0 | 0 | 0 | 9 | ratio |
| Example | 20 | 40 | 0 | 40 | 0 | 0 | 100 | wt % |
| 59 | 1 | 2 | 0 | 2 | 0 | 0 | 5 | ratio |
| Example | 14.286 | 57.143 | 0 | 28.571 | 0 | 0 | 100 | wt % |
| 60 | 1 | 4 | 0 | 2 | 0 | 0 | 7 | ratio |
| Example | 9.09 | 72.23 | 0 | 18.18 | 0 | 0 | 99.5 | wt % |
| 61 | 1 | 8 | 0 | 2 | 0 | 0 | 11 | ratio |
| Example | 12.85 | 51.41 | 10.03 | 25.71 | 0 | 0 | 100 | wt % |
| 62 | 1 | 4 | 0.8 | 2 | 0 | 0 | 7.8 | ratio |
| Example | 12.82 | 51.28 | 0 | 25.64 | 10.26 | 0 | 100 | wt % |
| 63 | 1 | 4 | 0 | 2 | 0.8 | 0 | 7.8 | ratio |
| Example | 11.39 | 45.56 | 10.02 | 22.78 | 10.25 | 0 | 100 | wt % |
| 64 | 1 | 4 | 0.9 | 2 | 0.9 | 0 | 8.8 | ratio |
| Example | 13.57 | 54.27 | 0 | 27.14 | 0 | 5.02 | 100 | wt % |
| 65 | 1 | 4 | 0 | 2 | 0 | 0.37 | 7.37 | ratio |
| Example | 14.29 | 57.14 | 0 | 28.57 | 0 | 0 | 100 | wt % |
| 66 | 1 | 4 | 0 | 2 | 0 | 0 | 7 | ratio |
| Example | 14.29 | 57.14 | 0 | 28.57 | 0 | 0 | 100 | wt % |
| 67 | 1 | 4 | 0 | 2 | 0 | 0 | 7 | ratio |

### Example 68

In a round bottom flask, 1 g of docetaxel is added to a solution of 4 g of PVP K30 dissolved in 50 mL of anhydrous ethanol. After stirring the solution for about 1 hour, 0.875 g of HPMC (6 mPas) and 0.875 g of poloxamer 407 are added, the solution is stirred for about 1 hour, and the solvent is removed from the solution thoroughly through a rotary evaporator. After adding 2 g of MgO to the entire obtained powders and mixing well, white powders are obtained.

### Example 69

In a round bottom flask, 1 g of docetaxel is added to a solution of 4 g of PVP K30 dissolved in 50 mL of anhydrous ethanol. After stirring the solution for about 1 hour, 0.875 g of HPMC (6 mPas) and 0.875 g of poloxamer 407 are added, the solution is stirred for about 1 hour, and the solvent is removed from the solution thoroughly through a rotary evaporator. After adding 2.5 g of MgO to the entire obtained powders and mixing well, white powders are obtained.

### Example 70

In a round bottom flask, 1 g of docetaxel is added to a solution of 4 g of PVP K30 dissolved in 50 mL of anhydrous ethanol. After stirring the solution for about 1 hour, 1 g of HPMC (6 mPas) and 1 g of poloxamer 407 are added, the solution is stirred for about 1 hour, and the solvent is removed from the solution thoroughly through a rotary evaporator. After adding 3 g of MgO to the entire obtained powders and mixing well, white powders are obtained.

### Example 71

In a round bottom flask, 1 g of docetaxel is added to a solution of 4 g of PVP K30 dissolved in 50 mL of anhydrous ethanol. After stirring the solution for about 1 hour, 0.368 g of polygamma glutamic acid (PGA) is added, the solution is stirred for about 1 hour, and the solvent is removed from the solution thoroughly through a rotary evaporator. After adding 2 g of MgO to the entire obtained powders and mixing well, white powders are obtained.

### Example 72

In a round bottom flask, 1 g of docetaxel is added to a solution of 4 g of PVP K30 dissolved in 50 mL of anhydrous ethanol. After stirring the solution for about 1 hour, 0.823 g of HPMC (6 mPas) and 0.412 g of polygamma glutamic acid (PGA) are added, the solution is stirred for about 1 hour, and the solvent is removed from the solution thoroughly through a rotary evaporator. After adding 2 g of MgO to the entire obtained powders and mixing well, white powders are obtained.

### Example 73

In a round bottom flask, 1 g of docetaxel is added to a solution of 4 g of PVP K30 dissolved in 50 mL of anhydrous ethanol. After stirring the solution for about 1 hour, 0.933 g of HPMC (6 mPas), 0.933 g of poloxamer 407, and 0.4467 g of polygamma glutamic acid (PGA) are added, the solution is stirred for about 1 hour, and the solvent is removed from the solution thoroughly through a rotary evaporator. After adding 2 g of MgO to the entire obtained powders and mixing well, white powders are obtained.

### Example 74

In a round bottom flask, 1 g of docetaxel is added to a solution of 4 g of PVP K30 dissolved in 50 mL of anhydrous ethanol. After stirring the solution for about 1 hour, 0.778 g of HPMC (100 mPas) is added, the solution is stirred for about 1 hour, and the solvent is removed from the solution thoroughly through a rotary evaporator. After adding 2 g of MgO to the entire obtained powders and mixing well, white powders are obtained.

### Example 75

In a round bottom flask, 1 g of docetaxel is added to a solution of 4 g of PVP K30 dissolved in 50 mL of anhydrous ethanol. After stirring the solution for about 1 hour, 0.875 g of HPMC (100 mPas) and 0.875 g of poloxamer 407 are added, the solution is stirred for about 1 hour, and the solvent is removed from the solution thoroughly through a rotary evaporator. After adding 2 g of MgO to the entire obtained powders and mixing well, white powders are obtained.

### Example 76

In a round bottom flask, 1 g of docetaxel is added to a solution of 4 g of PVP K30 dissolved in 50 mL of anhydrous ethanol. After stirring the solution for about 1 hour, 0.368 g of sodium dodecyl sulfate (SDS) is added, the solution is stirred for about 1 hour, and the solvent is removed from the solution thoroughly through a rotary evaporator. After adding 2 g of MgO to the entire obtained powders and mixing well, white powders are obtained.

### Example 77

In a round bottom flask, 1 g of docetaxel is added to a solution of 4 g of PVP K30 dissolved in 50 mL of anhydrous ethanol. After stirring the solution for about 1 hour, 0.823 g of HPMC (6 mPas) and 0.412 g of sodium dodecyl sulfate (SDS) are added, the solution is stirred for about 1 hour, and the solvent is removed from the solution thoroughly through a rotary evaporator. After adding 2 g of MgO to the entire obtained powders and mixing well, white powders are obtained.

### Example 78

In a round bottom flask, 1 g of docetaxel is added to a solution of 4 g of PVP K30 dissolved in 50 mL of anhydrous ethanol. After stirring the solution for about 1 hour, 0.933 g of HPMC (6 mPas), 0.933 g of poloxamer 407, and 0.467 g of sodium dodecyl sulfate (SDS) are added, the solution is stirred for about 1 hour, and the solvent is removed from the solution thoroughly through a rotary evaporator. After adding 2 g of MgO to the entire obtained powders and mixing well, white powders are obtained.

### Example 79

In a round bottom flask, 1 g of docetaxel is added to a solution of 4 g of PVP K30 dissolved in 50 mL of anhydrous ethanol. After stirring the solution for about 1 hour, 1 g of HPMC (6 mPas), 1 g of poloxamer 407, and 0.5 g of sodium dodecyl sulfate (SDS) are added, the solution is stirred for about 1 hour, and the solvent is removed from the solution thoroughly through a rotary evaporator. After adding 2.5 g of MgO to the entire obtained powders and mixing well, white powders are obtained.

### Example 80

In a round bottom flask, 1 g of docetaxel is added to a solution of 4 g of PVP K30 dissolved in 50 mL of anhydrous ethanol. After stirring the solution for about 1 hour, 0.179 g of MMT is added, the solution is stirred for about 1 hour, and the solvent is removed from the solution thoroughly through a rotary evaporator. After adding 2 g of MgO to the entire obtained powders and mixing well, white powders are obtained.

### Example 81

In a round bottom flask, 1 g of docetaxel is added to a solution of 4 g of PVP K30 dissolved in 50 mL of anhydrous ethanol. After stirring the solution for about 1 hour, 0.368 g of MMT is added, the solution is stirred for about 1 hour, and the solvent is removed from the solution thoroughly through a rotary evaporator. After adding 2 g of MgO to the entire obtained powders and mixing well, white powders are obtained.

### Example 82

In a round bottom flask, 1 g of docetaxel is added to a solution of 4 g of PVP K30 dissolved in 50 mL of anhydrous ethanol. After stirring the solution for about 1 hour, 0.903 g of HPMC (6 mPas), 0.903 g of poloxamer 407, and 0.226 g of MMT are added, the solution is stirred for about 1 hour, and the solvent is removed from the solution thoroughly through a rotary evaporator. After adding 2 g of MgO to the entire obtained powders and mixing well, white powders are obtained.

### Example 83

In a round bottom flask, 1 g of docetaxel is added to a solution of 4 g of PVP K30 dissolved in 50 mL of anhydrous ethanol. After stirring the solution for about 1 hour, 0.9 g of HPMC (6 mPas) and 0.9 g of poloxamer 407 are added, the solution is stirred for about 1 hour, the solvent is removed from the solution thoroughly through a rotary evaporator, and then 6.8 g of docetaxel-PVP-HPMC powders are obtained. After adding 2 g of MgO to the docetaxel-PVP-HPMC powders and mixing well, white powders are obtained.

### Comparative Example 1

Docetaxel 100 wt %

### Comparative Example 2

Taxotere

### Comparative Example 3

Vehicle control: sterile water for injection (Daihan Sterile Water for Injection)

### Experimental Example 1: In-Vivo Pharmacokinetic Analysis (Rat) of the Compositions of Examples 1 to 10 and 23 to 82, and Comparative Example 1

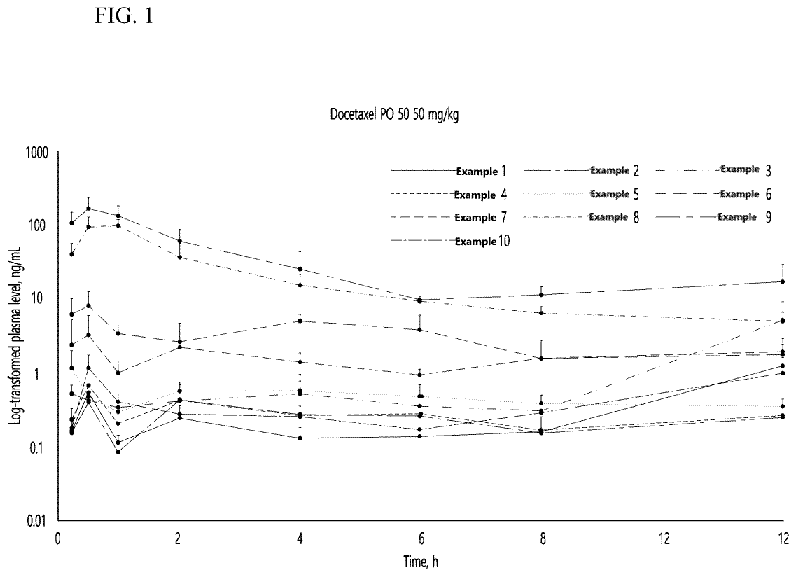

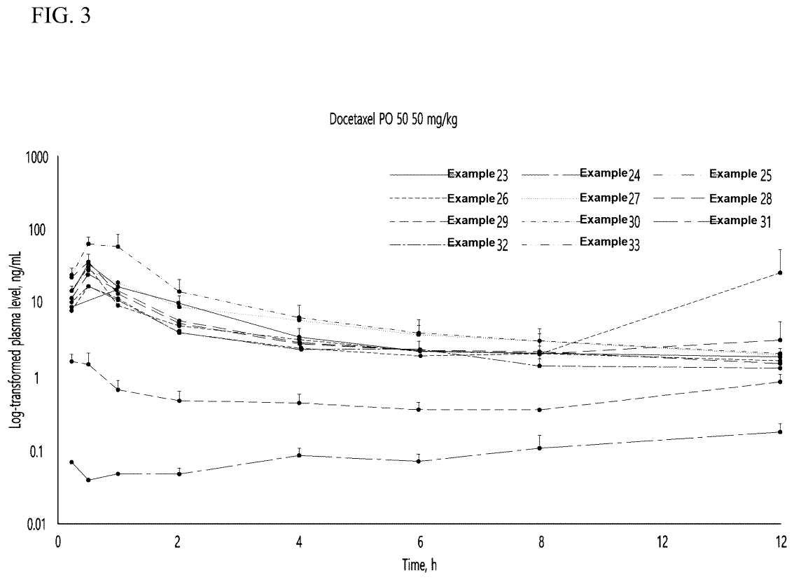

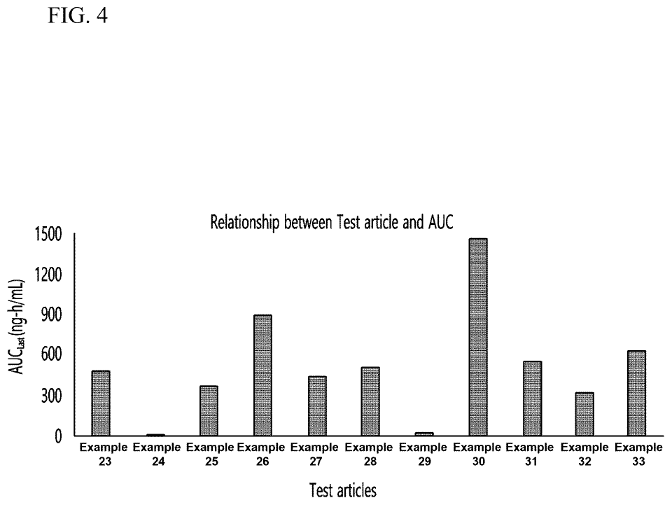

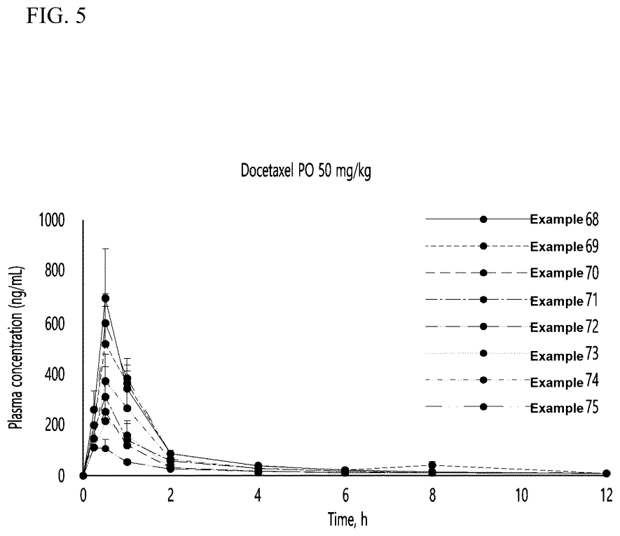

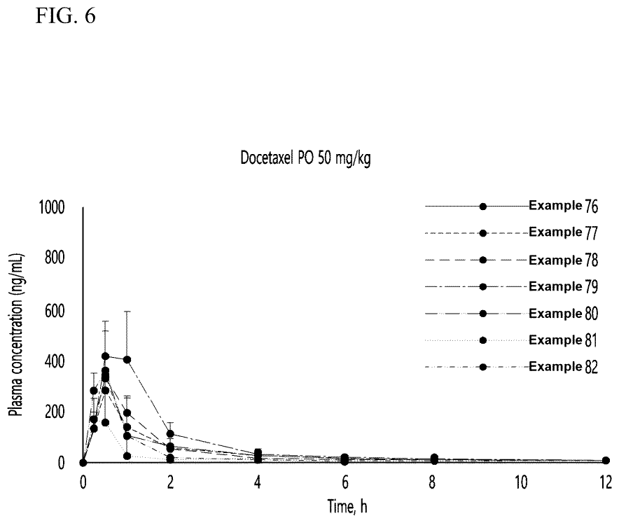

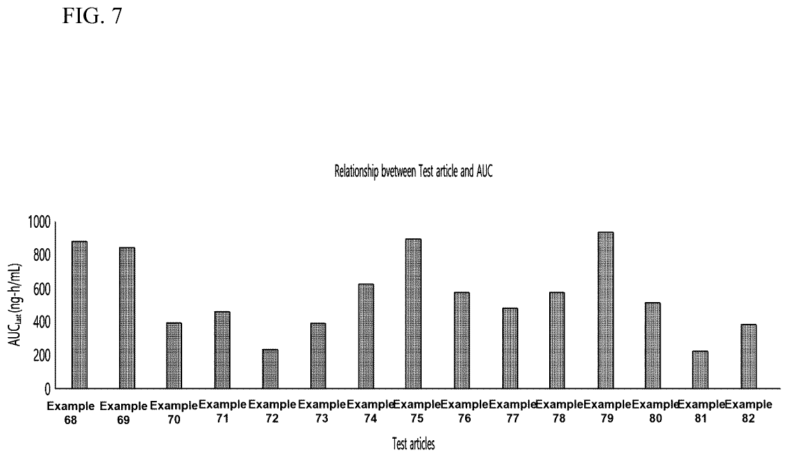

In vivo pharmacokinetic analysis was performed using the compositions of Examples 1 to 10 and 23 to 82, and Comparative Example 1. The compositions of Examples 1 to 10 and 23 to 82 and Comparative Example 1 were single orally administered to rats to obtain plasma drug concentration information. The results are shown in Tables 4 to 11 and FIGS. 1 to 7.

In addition, the experiment was conducted by administering the compositions of Examples 1 to 10 and 23 to 82 and the drug of Comparative Example 1 at a dose of 50 mg/kg, respectively.

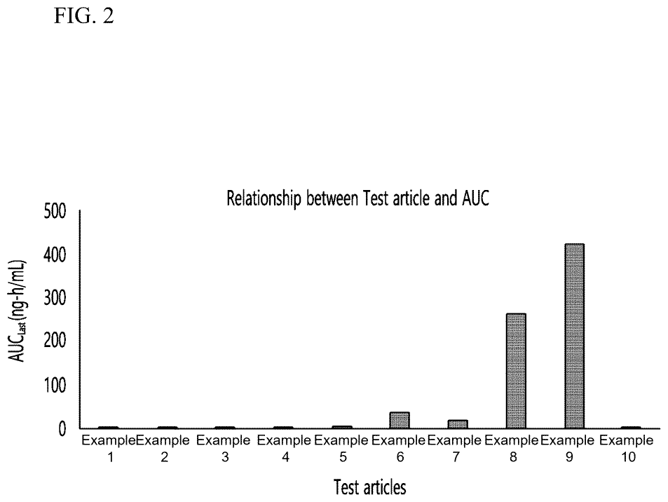

In the above analysis, the administration results of the compositions of Examples 1 to 10 and 23 to 82 and Comparative Example 1 are shown in FIGS. 1 to 7 and Tables 4 to 11. The graphs of FIGS. 1, 3, 5, and 6 show the docetaxel concentrations in plasma over time in rats, which were measured by collecting blood at 0 hr, 0.25 hr, 0.5 hr, 1 hr, 2 hr, 4 hr, 6 hr, 8 hr, and 12 hr. Specific pharmacokinetic parameters are described in Tables 5 and 7 to 9 below, and the relationships between the tested compositions and AUC are shown in FIGS. 2, 4, and 7.

The experimental results confirmed that when docetaxel was formulated with PVP, HPMC, poloxamer 407, alginate, polygamma glutamic acid, sodium dodecyl sulfate, PEG1400, or the like, the pharmacokinetic effect was improved by at least two times compared to the effect of conventional docetaxel administered orally. It was confirmed that when docetaxel was used in conjunction with MgO or MMT, the AUC value was improved by at least ten times, and the Cmax value could be improved by ten times or more. These can be immediately confirmed by numerically comparing the results of Comparative Example 1 and the results of Examples.

In addition, it was also confirmed that when the content of a polyvinyl pyrrolidone-based compound, a cellulose compound, a poloxamer-based compound, or polyethylene glycol was combined with docetaxel at a ratio lower than 1:1, the bioavailability was almost similar to that of docetaxel, so that the bioavailability was improved, but not as much as desired. Thus, it was confirmed that the content of each material also contributed to the improvement of the bioavailability.

The above experiment clearly confirmed that docetaxel having an anticancer effect could exhibit drug efficacy even in an oral formulation. Specifically, docetaxel is a second-generation taxoid-based anticancer drug that is most commonly used for breast, stomach, and lung cancer. Currently, a commercially available docetaxel product is an injection, Taxotere (Sanofi-Aventis Korea), which contains docetaxel and Tween 80 and is generally dissolved in distilled water for injection containing ethanol to be used. However, the product exhibits serious side effects caused by the drug itself and the solvent, and in particular, has fatal side effects, such as hypersensitivity and fluid retention caused by the solvent. To overcome these problems, an oral anticancer agent is being developed. The reason for low blood concentration of docetaxel after oral administration is that the drug's solubility and body permeability are very low. Accordingly, obtaining an oral bioavailability of most anticancer drugs, particularly drugs with excellent anticancer effects, such as taxoids (paclitaxel and docetaxel), is very difficult. More specifically, this is because taxoid-based anticancer drugs have little absorption/interaction in the gastrointestinal tract (GI). In the present invention, it was experimentally confirmed that anticancer/antiviral/anti-inflammatory treatment could be performed by increasing the blood concentration of the drug after oral administration of the above-mentioned poorly soluble substance to increase the drug's exposure to the body.

Therefore, the present invention can improve convenience and ease of use by formulating the poorly soluble drug for oral administration and also can reduce the cost of the drug. In addition, the injection form has the inconvenience of having to be administered under the supervision of a doctor or a nurse. The fact that it can be administered in an oral form can improve the efficiency for outpatient or home administration.

TABLE 4

| | Docetaxel PO 50 mg/kg | | | | | | | | | |
|---|---|---|---|---|---|---|---|---|---|---|
| Time | Example | Example | Example | Example | Example | Example | Example | Example | Example | Example |
| classification | 1 | 2 | 3 | 4 | 5 | 6 | 7 | 8 | 9 | 10 |
| h | DTX-A1 | DTX-A2 | DTX-A3 | DTX-A4 | DTX-A5 | DTX-A6 | DTX-A7 | DTX-A8 | DTX-A9 | DTX-A10 |
| 0 | ND | ND | ND | ND | ND | ND | ND | ND | ND | ND |
| 0.25 | 0.170 | 0.152 | 0.535 | 0.238 | 1.168 | 6.513 | 2.485 | 40.266 | 107.724 | 0.182 |
| 0.5 | 0.606 | 0.380 | 0.496 | 0.636 | 0.466 | 7.657 | 3.177 | 95.714 | 164.960 | 1.133 |
| 1 | 0.116 | 0.081 | 0.326 | 0.203 | 0.300 | 3.484 | 0.994 | 103.432 | 135.778 | 0.414 |
| 2 | 0.253 | 0.407 | 0.390 | 0.414 | 0.560 | 2.636 | 2.226 | 38.554 | 60.869 | 0.283 |
| 4 | 0.132 | 0.264 | 0.519 | 0.265 | 0.569 | 5.288 | 1.445 | 15.969 | 25.743 | 0.255 |
| 6 | 0.141 | 0.266 | 0.360 | 0.287 | 0.486 | 3.877 | 0.930 | 9.247 | 9.796 | 0.169 |
| 8 | 0.157 | 0.156 | 0.299 | 0.174 | 0.374 | 1.599 | 1.603 | 6.619 | 11.389 | 0.295 |
| 12 | 1.241 | 0.251 | 5.305 | 0.258 | 0.360 | 1.791 | 1.918 | 5.082 | 17.558 | 0.994 |

* Data are represented as the mean concentration (n = 3).
* Plasma concentration: ng/mL
* ND: not detected, BQL: below quantitative limit (<0.05 ng/mL)

TABLE 5

| | Docetaxel PO 50 mg/kg | | | | | | | | | |
|---|---|---|---|---|---|---|---|---|---|---|
| | Example | Example | Example | Example | Example | Example | Example | Example | Example | Example |
| PK | 1 | 2 | 3 | 4 | 5 | 6 | 7 | 8 | 9 | 10 |
| Parameters | DTX-A1 | DTX-A2 | DTX-A3 | DTX-A4 | DTX-A5 | DTX-A6 | DTX-A7 | DTX-A8 | DTX-A9 | DTX-A10 |
| AUC(last) | 1.86 | 2.86 | 3.21 | 3.21 | 5.48 | 37.77 | 19.29 | 261.82 | 422.29 | 3.55 |
| Cmax | 0.61 | 0.55 | 0.66 | 0.68 | 1.23 | 9.68 | 4.52 | 121.62 | 167.33 | 1.14 |
| Tmax | 0.50 | 0.50 | 0.50 | 0.50 | 0.25 | 0.50 | 2.00 | 0.50 | 0.50 | 0.50 |
| t_{1/2} | 5.99 | 6.19 | 4.44 | 7.22 | 10.28 | 8.57 | 7.13 | 4.66 | 3.24 | 7.60 |

* AUC: ng · h/mL, Cₘₐₓ: ng/mL, Tₘₐₓ & t_{1/2}: h
** NC: Not Calculated

TABLE 6

| | Docetaxel PO 50 mg/kg | | | | | | | | | | |
|---|---|---|---|---|---|---|---|---|---|---|---|
| | Example | Example | Example | Example | Example | Example | Example | Example | Example | Example | Example |
| PK | 23 | 24 | 25 | 26 | 27 | 28 | 29 | 30 | 31 | 32 | 33 |
| Parameters | DTX-B1 | DTX-B2 | DTX-B3 | DTX-B4 | DTX-B5 | DTX-B6 | DTX-B7 | DTX-B8 | DTX-B9 | DTX-B10 | DTX-B11 |
| AUC(last) | 435.93 | 2.27 | 277.06 | 856.18 | 365.60 | 418.85 | 22.46 | 1410.24 | 476.31 | 255.11 | 589.24 |
| Cmax | 224.96 | 0.43 | 147.95 | 491.16 | 274.60 | 270.66 | 8.36 | 1112.17 | 453.91 | 150.67 | 332.60 |
| Tmax | 0.50 | 12.00 | 0.50 | 0.50 | 0.50 | 0.50 | 0.25 | 0.50 | 0.50 | 0.50 | 0.50 |
| t_{1/2} | 4.07 | NC | 3.87 | 4.07 | 4.40 | 5.75 | 6.10 | 6.11 | 5.44 | 3.53 | 3.49 |

* AUC: ng · h/mL, Cₘₐₓ: ng/mL, Tₘₐₓ & t_{1/2}: h
** NC: Not Calculated

TABLE 7

| | Docetaxel PO 50 mg/kg | | | | | | | | | |
|---|---|---|---|---|---|---|---|---|---|---|
| PK | Example | Example | Example | Example | Example | Example | Example | Example | Example | Example |
| Parameters | 34 | 35 | 36 | 37 | 38 | 39 | 40 | 41 | 42 | 43 |
| AUC(last) | 1.86 | 2.86 | 3.21 | 3.21 | 5.48 | 37.77 | 19.29 | 261.82 | 422.29 | 3.55 |
| Cmax | 0.61 | 0.55 | 0.66 | 0.68 | 1.23 | 9.68 | 4.52 | 121.62 | 167.33 | 1.14 |
| Tmax | 0.5 | 0.5 | 0.5 | 0.5 | 0.25 | 0.5 | 2 | 0.5 | 0.5 | 0.5 |
| t_{1/2} | 5.99 | 6.19 | 4.44 | 7.22 | 10.28 | 8.57 | 7.13 | 4.66 | 3.24 | 7.6 |

* AUC: ng · h/mL, Cmax: ng/mL, Tmax & t½: h
** NC: Not Calculated

TABLE 8

| | Docetaxel PO 50 mg/kg | | | | | | | | | | | | |
|---|---|---|---|---|---|---|---|---|---|---|---|---|---|
| PK | Comparative | Exam- | Exam- | Exam- | Exam- | Exam- | Exam- | Exam- | Exam- | Exam- | Exam- | Exam- | Exam- |
| Parameters | Example 1 | ple 44 | ple 45 | ple 46 | ple 47 | ple 48 | ple 49 | ple 50 | ple 51 | ple 52 | ple 53 | ple 54 | ple 55 |
| AUC(last) | 0.85 | 187.33 | 55.66 | 210.95 | 1.29 | 2.33 | 5.88 | 2.42 | 1.31 | 2.57 | 2.39 | 0.94 | 1.81 |
| Cmax | 0.12 | 60.05 | 34.68 | 63.34 | 0.23 | 0.37 | 1.6 | 0.43 | 0.34 | 0.44 | 0.47 | 0.16 | 0.37 |
| Tmax | 12 | 0.5 | 0.25 | 0.5 | 12 | 8 | 12 | 4 | 12 | 8 | 8 | 8 | 12 |
| t_{1/2} | NC | 3.94 | 3.84 | 4.52 | NC | NC | NC | NC | 9.26 | 10.24 | 12.93 | 3.28 | NC |

* AUC: ng · h/mL, Cmax: ng/mL, Tmax & t½: h
** NC: Not Calculated

TABLE 9

| | Docetaxel PO 50 mg/kg | | | | | | | | | | | |
|---|---|---|---|---|---|---|---|---|---|---|---|---|
| PK | Exam- | Exam- | Exam- | Exam- | Exam- | Exam- | Exam- | Exam- | Exam- | Exam- | Exam- | Exam- |
| Parameters | ple 56 | ple 57 | ple 58 | ple 59 | ple 60 | ple 61 | ple 62 | ple 63 | ple 64 | ple 65 | ple 66 | ple 67 |
| AUC(last) | 5.47 | 435.93 | 2.27 | 277.06 | 856.18 | 365.6 | 418.85 | 22.46 | 1410.24 | 476.31 | 255.11 | 589.24 |
| Cmax | 1.74 | 224.96 | 0.43 | 147.95 | 491.16 | 274.6 | 270.66 | 8.36 | 1112.17 | 453.91 | 150.67 | 332.6 |
| Tmax | 8 | 0.5 | 12 | 0.5 | 0.5 | 0.5 | 0.5 | 0.25 | 0.5 | 0.5 | 0.5 | 0.5 |
| t_{1/2} | 7.53 | 4.07 | NC | 3.87 | 4.07 | 4.4 | 5.75 | 6.1 | 6.11 | 5.44 | 3.53 | 3.49 |

* AUC: ng · h/mL, Cmax: ng/mL, Tmax & t½: h
** NC: Not Calculated

TABLE 10

| | Docetaxel PO 50 mg/kg | | | | | | | |
|---|---|---|---|---|---|---|---|---|
| PK | Example | Example | Example | Example | Example | Example | Example | Example |
| Parameters | 68 | 69 | 70 | 71 | 72 | 73 | 74 | 75 |
| AUC(last) | 877.76 | 845.29 | 392.60 | 464.46 | 238.17 | 394.62 | 623.50 | 899.64 |
| Cmax | 692.71 | 530.04 | 247.20 | 303.70 | 109.29 | 226.83 | 369.69 | 593.97 |
| Tmax | 0.50 | 0.50 | 0.50 | 0.50 | 0.25 | 0.50 | 0.50 | 0.50 |
| t_{1/2} | 2.11 | 2.65 | 2.89 | 2.26 | 2.95 | 3.68 | 3.72 | 3.50 |

| | Docetaxel PO 50 mg/kg | | | | | | |
|---|---|---|---|---|---|---|---|
| PK | Example | Example | Example | Example | Example | Example | Example |
| Parameters | 76 | 77 | 78 | 79 | 80 | 81 | 82 |
| AUC(last) | 570.08 | 481.17 | 577.36 | 939.19 | 752.64 | 359.62 | 507.59 |
| Cmax | 400.53 | 311.01 | 348.23 | 477.28 | 479.27 | 243.89 | 371.64 |
| Tmax | 0.50 | 0.50 | 0.50 | 0.50 | 0.50 | 0.25 | 0.50 |
| t_{1/2} | 3.18 | 3.57 | 3.33 | 4.41 | 6.84 | 4.32 | 3.72 |

* AUC: ng · h/mL, Cmax: ng/mL, Tmax & t½: h
** NC: Not Calculated

TABLE 11

| | Docetaxel PO 50 mg/kg | | | | | | | | | | | | | | |
|---|---|---|---|---|---|---|---|---|---|---|---|---|---|---|---|
| Time | Exam- | Exam- | Exam- | Exam- | Exam- | Exam- | Exam- | Exam- | Exam- | Exam- | Exam- | Exam- | Exam- | Exam- | Exam- |
| h | ple 68 | ple 69 | ple 70 | ple 71 | ple 72 | ple 73 | ple 74 | ple 75 | ple 76 | ple 77 | ple 78 | ple 79 | ple 80 | ple 81 | ple 82 |
| 0 | ND | ND | ND | ND | ND | ND | ND | ND | ND | ND | ND | ND | ND | ND | ND |
| 0.25 | 260.897 | 166.097 | 162.574 | 150.757 | 107.301 | 105.470 | 146.886 | 199.772 | 310.947 | 153.670 | 182.744 | 171.119 | 138.396 | 140.019 | 145.820 |
| 0.5 | 692.710 | 514.667 | 247.200 | 303.704 | 107.038 | 211.701 | 369.690 | 593.967 | 400.201 | 311.005 | 348.235 | 461.267 | 419.069 | 230.914 | 371.639 |
| 1 | 341.427 | 362.090 | 120.355 | 139.667 | 55.156 | 155.783 | 264.428 | 382.465 | 118.194 | 158.198 | 216.142 | 449.490 | 299.554 | 121.807 | 199.974 |
| 2 | 86.551 | 69.120 | 35.004 | 57.514 | 28.217 | 35.871 | 67.751 | 88.460 | 61.373 | 62.070 | 60.983 | 123.789 | 110.591 | 35.157 | 45.140 |
| 4 | 32.070 | 30.306 | 15.543 | 21.499 | 14.489 | 16.680 | 27.426 | 39.355 | 22.048 | 24.638 | 28.159 | 38.995 | 37.624 | 20.308 | 25.569 |
| 6 | 19.175 | 22.421 | 8.548 | 12.123 | 8.246 | 11.527 | 17.501 | 21.927 | 21.045 | 11.556 | 18.092 | 24.972 | 17.325 | 9.285 | 12.222 |
| 8 | 13.774 | 37.887 | 19.178 | 13.987 | 10.939 | 16.710 | 14.009 | 21.088 | 19.913 | 9.886 | 13.087 | 17.992 | 13.511 | 8.389 | 9.269 |
| 12 | 11.530 | 8.201 | 5.384 | 6.326 | 4.613 | 5.839 | 8.158 | 10.797 | 7.818 | 6.165 | 7.873 | 9.361 | 9.953 | 4.790 | 5.344 |

* Data are represented as the mean concentration (n = 3).
* Plasma concentration: ng/mL
* ND: not detected, BQL: below quantitative limit (<0.05 ng/mL)

### Experimental Example 2: In-Vivo Pharmacokinetic Analysis (Beagle) of the Compositions of Example 83 and Comparative Example 1

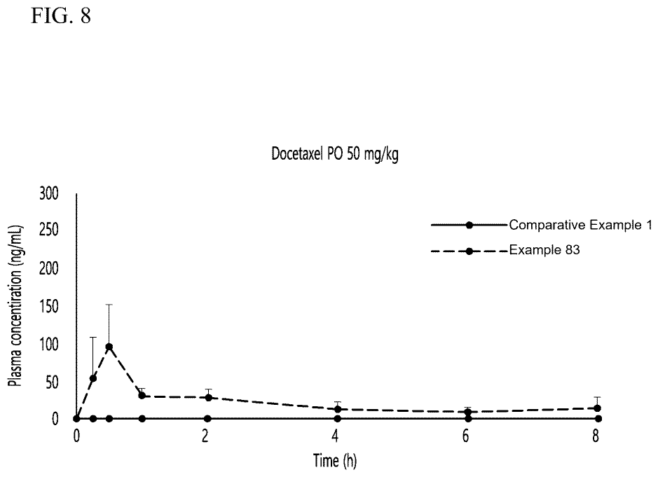

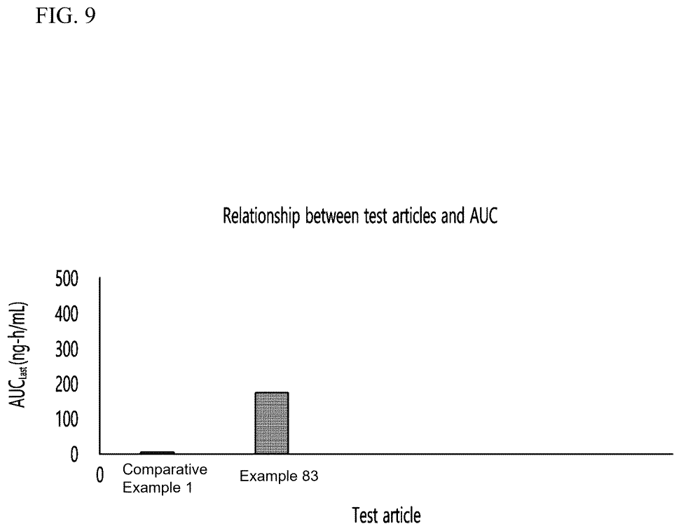

In vivo pharmacokinetic analysis was performed using the compositions of Example 83 and Comparative Example 1. The compositions of Example 83 and Comparative Example 1 were single orally administered to beagles to obtain plasma drug concentration information. The results are shown in Tables 12 and 13 below and FIGS. 8 and 9.

In addition, the experiment was conducted by administering the composition of Example 83 and the drug of Comparative Example 1 at a dose of 50 mg/kg, respectively.

The graph of FIG. 8 shows the docetaxel concentrations in plasma over time in beagles, which were measured by collecting blood at 0 hr, 0.25 hr, 0.5 hr, 1 hr, 2 hr, 4 hr, 6 hr, and 8 hr. Specific pharmacokinetic parameters are described in Table 13 below and FIG. 8, and the relationships between the tested compositions and AUC are shown in Table 12 and FIG. 9.

The experimental results confirmed that when docetaxel was formulated with compounds such as PVP, HPMC, and MgO, the pharmacokinetic effect was improved by at least 100 times compared to the effect of conventional docetaxel administered orally. This can be immediately confirmed by numerically comparing the results of Comparative Example 1 and Example 83.

The above experiment confirmed that docetaxel having an anticancer effect could exhibit drug efficacy even in an oral formulation. In particular, it was confirmed that the pharmacokinetic effect of the formulated docetaxel was remarkably improved even in the form of an oral formulation, even in individuals with a relatively large body and excessive secretion of gastric juice, such as beagles, compared to rats.

Therefore, the present invention can improve convenience and ease of use by formulating the poorly soluble drug for oral administration and also can reduce the cost of the drug. In addition, the injection form has the inconvenience of having to be administered under the supervision of a doctor or a nurse. The fact that it can be administered in an oral form can improve the efficiency for outpatient or home administration.

TABLE 12

| | Docetaxel PO 50 mg/kg | |
|---|---|---|
| | Comparative | |
| PK Parameters | Example 1 | Example 83 |
| AUC (last) | 1.86 | 173.34 |
| Cmax | 0.50 | 112.98 |
| Tmax | 3.50 | 0.38 |
| t_{1/2} | NC | 3.46 |

* AUC: ng · h/mL, Cmax: ng/mL, Tmax & t_{1/2}: h

TABLE 13

| | Docetaxel PO 50 mg/kg | |
|---|---|---|
| Time | Comparative | |
| h | Example 1 | Example 83 |
| 0 | ND | ND |
| 0.25 | 0.239 | 53.817 |
| 0.5 | 0.219 | 94.890 |
| 1 | 0.243 | 29.472 |
| 2 | 0.140 | 28.140 |
| 4 | 0.621 | 12.834 |
| 6 | 0.675 | 9.781 |
| 8 | 0.222 | 14.757 |

* Data are represented as the mean concentration (n = 2).
* Plasma concentration: ng/mL
*ND: not detected

### Experimental Example 3: Determination of the Anticancer Effects of the Compositions of Example 78, Comparative Example 2, and Control Example 1

Anticancer effect analysis was performed using the compositions of Example 78, Comparative Example 2, and Control Example 1. A method of administering Example 78, Comparative Example 2, and Control Example 1 to mice was used, and the conditions of the mice are shown in Table 14 below.

TABLE 14

| | |
|---|---|
| Species and strain | BALB/c nude mice, BALB/cAJcl-nu/nu |
| Producer | CLEA Inc. (Japan) |
| Supplier | Saeron Bio (800-17 Cheonggye-dong, |
| | Uiwang-si, Gyeonggi-do) |
| Gender | Female |
| Age | 6 weeks old |
| Number of animals | 120 animals |
| acquired | |
| Number of animals | 66 animals |
| used | |
| Identification | Tail-marking method |

Pancreas carcinoma was induced by transplanting a cell line (PANC-1) used in a cancer xenograft model into the mice. Specifically, one vial of frozen human tumor cell line (PANC-1 cell line) was added to a cell culture flask together with DMEM medium (Welgene, LM01201805) containing 10% heat-inactivated FBS (fetal bovine serum, Gibco, 10082-147) and incubated at a 37° C., 5% CO2 incubator. After washing with PBS, 2.5% trypsin-EDTA (Gibco, 15090) was diluted 10 times, and cells were separated by adding the diluted 2.5% trypsin-EDTA. Centrifugation (1,000 rpm, 5 minutes) was performed, the supernatant was discarded, and a cell suspension was obtained with a new medium. The viability was determined under a microscope, and a cell line was prepared by diluting cells with DPBS at a concentration of 2.0×10⁷ cells/mL. After the adaptation period in the breeding environment of mice was over, the dorsal regions of the mice were disinfected with 70% alcohol and then administered an subcutaneously at administration amount of 2.0×106 cells/0.1 mL/head using a syringe equipped with a 26 gauge needle. The cancer-induced mice were grouped as shown in Table 15 below, and the administration content and administration route for each group were indicated.

TABLE 15

| | | Number of | | | Dose | Administration | | |
|---|---|---|---|---|---|---|---|---|
| | | animals | Animal | Administered | (mg/kg | volume | Number of | Route of |
| Group | Gender | (heads) | number | material | P.O,) | (mL/kg) | administrations | administration |
| G1 | F | 6 | 1-6 | Vehicle | — | 10 | — | PO |
| G2 | F | 6 | 7-12 | Taxotere | 2 | 10 | Three times | IV |
| | | | | | | | (Day 0, 3, 6) | |
| G3 | F | 6 | 13-18 | Example 78 | 10 | 10 | Twice/day | PO |
| G4 | F | 6 | 19-24 | Example 78 | 10 | 10 | Three | PO |
| | | | | | | | times/day | |
| G5 | F | 6 | 25-30 | Example 78 | 15 | 10 | Twice/day | PO |
| G6 | F | 6 | 31-36 | Example 78 | 15 | 10 | Three | PO |
| | | | | | | | times/day | |
| G7 | F | 6 | 37-42 | Example 78 | 20 | 10 | Four | PO |
| | | | | | | | times/week | |
| G8 | F | 6 | 43-48 | Example 78 | 20 | 10 | Once/day | PO |
| G9 | F | 6 | 49-54 | Example 78 | 20 | 10 | Twice/day | PO |
| G10 | F | 6 | 55-60 | Example 78 | 40 | 10 | Four | PO |
| | | | | | | | times/week | |
| G11 | F | 6 | 61-66 | Example 78 | 50 | 10 | Four | PO |
| | | | | | | | times/week | |

Specifically, for G2, the test material was administered on the start date of administration (Day 0) and then on the third day (Day 3) and the sixth day (Day 6) after the test material was administered (8:00). For G3, G5 and G9, the test material was administered twice/day for 2 weeks (8:00 and 18:00). For G4 and G6, the test material was administered three times/day for 2 weeks (8:00, 13:00 and 18:00). For G7, G10, and G11, the test material was administered on the start date of administration (Day 0), and then once/two days, for 2 weeks (8:00). For G8, the test material was administered once/day for 2 weeks (8:00).

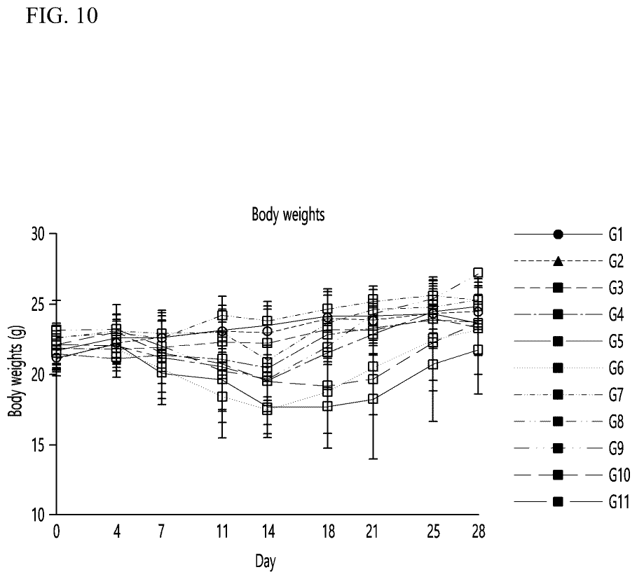

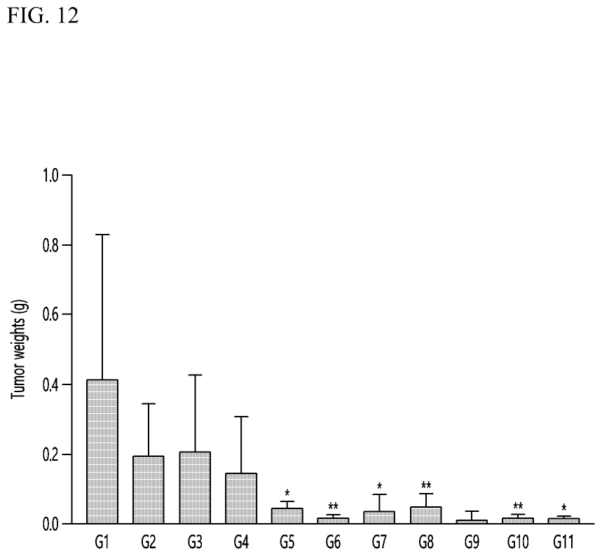

During the observation period after administration as above and during administration and observation periods, the type of general symptoms, including death, the date of incidence, and the degree of symptoms were observed once a day and recorded for each individual. Individuals with worsening general symptoms were isolated, and moribund and dead animals were handled, conforming with the scheduled necropsy animals. Their body weight and tumor size measurements were compared and described in FIGS. 10 and 12.
Body weight measurement criteria: Measurements were taken on the day of group separation, twice a week afterwards, and on the day of necropsy.
Tumor size measurement: On the day of group separation and start of administration of the test material (Day 0) and twice a week afterwards for four weeks, tumor occurrence was determined, and tumor size was measured. Major and minor axes of the tumor were measured by using calipers, and the tumor size was calculated by using the following formula: Tumor size=ab2/2 (a: major axis length, b: minor axis length)
Necropsy: On the day of the necropsy, all surviving animals were anesthetized, the abdominal aorta and the postcaval vein were cut to exsanguinate/kill the animal, and then tumors were extracted. The extracted tumors were lightly washed with PBS, drained, and photographed, and the weight was measured.

As a result of general symptom observation, one individual from administration group G5 died on the eighth day after the administration start of the test material, and one from administration group G11 was dead on the tenth day after the administration start of the test material. On the 12th day after the administration start of the test material, one individual from administration G6 group and one from from administration group G9 were dead. Two individuals administration group G9 died on the 13th day after the administration start of the test material, and one from G9 was dead on the 15th day after the administration start of the test material. Two individuals from G11 died on the 18th day after the administration start of the test material, and one from administration group G7 was dead on the 20th day after the administration start of the test material.

As a result of the body weight measurement, the weight levels of G6, G9, and G11 were significantly lower than that of G1 on the 11th day after the administration start of the test material (p<0.01 or p<0.05), and on the 14th day after the administration start of the test material, the weight levels of G5, G6, Example 78 40 mg/kg P.O, four times/week P.O administration group (G10) and G11 were significantly lower than that of vehicle control (G1) (p<0.001 [symbol] p<0.05). The weight levels of G6, G10, and G11 were significantly lower than that of G1 on the 18th day after the administration start of the test material (p<0.001 or p<0.01), and on the 21st day after the administration start of the test material, the weight levels of G10 and G11 were significantly lower than that of G1 (p<0.01).

As a result of the tumor size measurement, the tumor size level of G10 was significantly lower than that of G1 from the 11th to the 28th day after the administration start of the test material (p<0.05), and from the 18th to the 28th day after the administration start of the test material, the tumor size levels of G5, G6, G7, Example 78 20 mg/kg P.O, once/day P.O administration group (G8), G10 and G11 were significantly lower than that of G1 (p<0.01 or p<0.05), and from the 21st to the 28th day after the administration start of the test material, the tumor size level of G11 was significantly lower than that of G1 (p<0.05).

As a result of the tumor weight measurement, the tumor weight levels of G5, G6, G7, G8, G10, and G11 were significantly lower than that of G1 (p<0.01 or p<0.05).

This test was conducted to evaluate anticancer effects by subcutaneously transplanting PANC-1 cells, a human tumor cell line, into nude mice to construct a xenograft model and then administering the test material (G1: Vehicle control, G2: Taxotere 2 mg/kg P.O, I.V administration group, G3: Example 78 10 mg/kg P.O, twice/day administration group, G4: Example 78 10 mg/kg P.O, three times/day administration group, G5: Example 78 15 mg/kg P.O, twice/day administration group, G6: Example 78 15 mg/kg P.O, three times/day administration group, G7: Example 78 20 mg/kg P.O, four times/week administration group, G8: Example 78 20 mg/kg P.O, once/day administration group, G9: Example 78 20 mg/kg P.O, twice/day administration group, G10: Example 78 40 mg/kg P.O, four times/week administration group, G11: Example 78 50 mg/kg P.O, four times/week administration group).

As a result of general symptom observation, one individual from Example 78 15 mg/kg P.O, twice/day administration group, one individual from Example 78 15 mg/kg P.O, three times/day administration group, one individual from Example 78 20 mg/kg P.O, four times/week administration group, four individuals from Example 78 20 mg/kg P.O, twice/day administration group, and three individuals from Example 78 50 mg/kg P.O, four times/week administration group were dead. The result is considered to be due to the high concentration administration or multiple administrations of the test material.

As a result of the body weight measurement, from the 11th to the 21st day after the test material administration, the weight levels of some of the test material administration groups were significantly reduced compared to that of the induced control group. Afterward, statistically significant differences were not observed, but a dose-correlated decreasing trend was observed compared to the induced control group. Thus, the corresponding weight change is considered to be due to a change caused by the test material.

As a result of the tumor size measurement, from the 18th to the 28th day after the test material administration, the tumor size levels of the test material groups (G3-G11), except for Example 78 10 mg/kg P.O, twice/day administration group, Example 78 10 mg/kg P.O, three times/day administration group, and Example 78 20 mg/kg P.O, twice/day administration group, were observed to be significantly lower than that of the induced control group, and a dose-correlated decreasing trend was observed.

As a result of the tumor weight measurement, the tumor weight levels of the test material groups (G3-G11), except for Example 78 10 mg/kg P.O, twice/day administration group and Example 78 10 mg/kg P.O, three times/day administration group, were observed to be significantly lower than that of the induced control group. From the result, it is considered that when the concentration of the test material Example 78 exceeded 10 mg/kg, it could help reduce the size and weight of the tumor.

Under the present test conditions, it was observed that when the test material Example 78 was administered to the Xenograft Model, which was prepared by subcutaneously transplanting PANC-1 cells into nude mice, the size and weight levels of the tumors of other concentration administration groups than Example 78 10 mg/kg administration group were statistically lower than those of the induced control group, and a dose-dependent trend was observed. However, dead animals were observed in the groups other than Example 78 20 mg/kg P. O, once/day administration group and Example 78 40 mg/kg P.O, four times/week administration group.

Therefore, it is considered that administration of 20 mg/kg once/day and 40 mg/kg four times/week of the test material Example 78 to the xenograft model, prepared by subcutaneously transplanting the PANC-1 cell line into nude mice, exhibited an anticancer effect against PANC-1 cells.

1. A pharmaceutical composition comprising: docetaxel or a pharmaceutically acceptable salt thereof; and one or more compounds selected from among a polyvinyl pyrrolidone-based compound, a cellulose-based compound, a poloxamer-based compound, a polyethylene glycol-based compound, an alginic acid-based compound, polygamma glutamic acid and sodium dodecyl sulfate.

2. The pharmaceutical composition of claim 1, further comprising one or more compounds selected from among a crystallization inhibitor, a swellable polymer, an enteric coating, a foam generator, and a swellable excipient.

3. The pharmaceutical composition of claim 1, wherein the polyvinyl pyrrolidone-based compound is one or more selected from the group consisting of polyvinylpyrrolidone K10 (MW 8000-10,000), polyvinylpyrrolidone K12 (MW 11,000-12,000), polyvinylpyrrolidone K15 (MW 14,000-18,000), polyvinylpyrrolidone K17 (MW 14,000-18,000), polyvinylpyrrolidone K18 (MW 14,000-18,000), polyvinylpyrrolidone K25 (MW 2,000-25,000), polyvinylpyrrolidone K30 (MW3,000-40,000), polyvinylpyrrolidone K60 (MW 5,000-60,000), and polyvinylpyrrolidone K90 (MW 8,000-90,000).

4. The pharmaceutical composition of claim 1, wherein the poloxamer-based compound is one or more selected from the group consisting of poloxamer 101, poloxamer 105, poloxamer 105 benzoate, poloxamer 108, poloxamer 122, poloxamer 123, poloxamer 124, poloxamer 181, poloxamer 182, poloxamer 182 dibenzoate, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

5. The pharmaceutical composition of claim 1, wherein the cellulose-based compound is one or more selected from the group consisting of hydroxypropyl methylcellulose (HPMC), hydroxyethyl cellulose, hydroxypropyl cellulose (HPC), carboxymethylcellulose (CMC), ethylcellulose (EC), methylcellulose (MC) and cellulose acetate (CA).

6. The pharmaceutical composition of claim 1, wherein the polyethylene glycol-based compound is one or more selected from the group consisting of polyethylene glycol 200, polyethylene glycol300, polyethylene glycol 400, polyethylene glycol 500, polyethylene glycol 1000, polyethylene glycol 1400, polyethylene glycol 1500, polyethylene glycol 4000, polyethylene glycol 8000, polyethylene glycol 10000, and methoxy polyethylene glycol 550.

7. The pharmaceutical composition of claim 1, which is one or more selected from among: a 1st combination comprising docetaxel or a pharmaceutically acceptable salt thereof and a polyvinyl pyrrolidone-based compound; a 2nd combination comprising docetaxel or a pharmaceutically acceptable salt thereof and a cellulose-based compound; a 3rd combination comprising docetaxel or a pharmaceutically acceptable salt thereof and a poloxamer-based compound; a 4th combination comprising docetaxel or a pharmaceutically acceptable salt thereof and a polyethylene glycol-based compound; a 5th combination comprising docetaxel or a pharmaceutically acceptable salt thereof and an alginic acid-based compound; a 6th combination comprising docetaxel or a pharmaceutically acceptable salt thereof and polygamma glutamic acid; a 7th combination comprising docetaxel or a pharmaceutically acceptable salt thereof and sodium dodecyl sulfate; an 8th combination comprising docetaxel or a pharmaceutically acceptable salt thereof, a polyvinyl pyrrolidone-based compound, and a cellulose-based compound; a 9th combination comprising docetaxel or a pharmaceutically acceptable salt thereof, a polyvinyl pyrrolidone-based compound, and a poloxamer-based compound; a 10th combination comprising docetaxel or a pharmaceutically acceptable salt thereof, a polyvinyl pyrrolidone-based compound, and a polyethylene glycol-based compound; an 11th combination comprising docetaxel or a pharmaceutically acceptable salt thereof, a polyvinyl pyrrolidone-based compound, and an alginic acid-based compound; a 12th combination comprising docetaxel or a pharmaceutically acceptable salt thereof, a polyvinyl pyrrolidone-based compound, and polygamma glutamic acid; a 13th combination comprising docetaxel or a pharmaceutically acceptable salt thereof, a polyvinyl pyrrolidone-based compound, and sodium dodecyl sulfate; a 14th combination comprising docetaxel or a pharmaceutically acceptable salt thereof, a cellulose-based compound, and a poloxamer-based compound; a 15th combination comprising docetaxel or a pharmaceutically acceptable salt thereof, a cellulose-based compound, and a polyethylene glycol-based compound; a 16th combination comprising docetaxel or a pharmaceutically acceptable salt thereof, a cellulose-based compound, and an alginic acid-based compound; a 17th combination comprising docetaxel or a pharmaceutically acceptable salt thereof, a cellulose-based compound and polygamma glutamic acid; an 18th combination comprising docetaxel or a pharmaceutically acceptable salt thereof, a cellulose-based compound, and sodium dodecyl sulfate; a 19th combination comprising docetaxel or a pharmaceutically acceptable salt thereof, a poloxamer-based compound, and a polyethylene glycol-based compound; a 20th combination comprising docetaxel or a pharmaceutically acceptable salt thereof, a poloxamer-based compound, and an alginic acid-based compound; a 21st combination comprising docetaxel or a pharmaceutically acceptable salt thereof, a poloxamer-based compound, and polygamma glutamic acid; a 22nd combination comprising docetaxel or a pharmaceutically acceptable salt thereof, a poloxamer-based compound and sodium dodecyl sulfate; a 23rd combination comprising docetaxel or a pharmaceutically acceptable salt thereof, a polyethylene glycol-based compound, and an alginic acid-based compound; a 24th combination comprising docetaxel or a pharmaceutically acceptable salt thereof, a polyethylene glycol compound, and polygamma glutamic acid; a 25th combination comprising docetaxel or a pharmaceutically acceptable salt thereof, a polyethylene glycol-based compound, and sodium dodecyl sulfate; a 26th combination comprising docetaxel or a pharmaceutically acceptable salt thereof, an alginic acid-based compound, and polygamma glutamic acid; a 27th combination comprising docetaxel or a pharmaceutically acceptable salt thereof, polygamma glutamic acid, and sodium dodecyl sulfate; a 28th combination comprising docetaxel or a pharmaceutically acceptable salt thereof, polygamma glutamic acid, and sodium dodecyl sulfate; a 29th combination comprising docetaxel or a pharmaceutically acceptable salt thereof, a polyvinyl pyrrolidone-based compound, a cellulose-based compound, and a poloxamer-based compound; a 30th combination comprising docetaxel or a pharmaceutically acceptable salt thereof, a polyvinyl pyrrolidone-based compound, a cellulose-based compound, and a polyethylene glycol-based compound; a 31st combination comprising docetaxel or a pharmaceutically acceptable salt thereof, a polyvinyl pyrrolidone-based compound, a cellulose-based compound, and an alginic acid-based compound; a 32nd combination comprising docetaxel or a pharmaceutically acceptable salt thereof, a polyvinyl pyrrolidone-based compound, a cellulose-based compound, and poly-gamma glutamic acid; a 33rd combination comprising docetaxel or a pharmaceutically acceptable salt thereof, a polyvinyl pyrrolidone-based compound, a cellulose-based compound, and sodium dodecyl sulfate; a 34th combination comprising docetaxel or a pharmaceutically acceptable salt thereof, a polyvinyl pyrrolidone-based compound, a poloxamer-based compound, and a polyethylene glycol-based compound; a 35th combination comprising docetaxel or a pharmaceutically acceptable salt thereof, a polyvinyl pyrrolidone-based compound, a poloxamer-based compound, and an alginic acid-based compound; a 36th combination comprising docetaxel or a pharmaceutically acceptable salt thereof, a polyvinyl pyrrolidone-based compound, a poloxamer-based compound, and sodium dodecyl sulfate; a 37th combination comprising docetaxel or a pharmaceutically acceptable salt thereof, a polyvinyl pyrrolidone-based compound, a polyethylene glycol-based compound, and an alginic acid-based compound; a 38th combination comprising docetaxel or a pharmaceutically acceptable salt thereof, a polyvinyl pyrrolidone-based compound, a polyethylene glycol-based compound, and polygamma glutamic acid; a 38th combination comprising docetaxel or a pharmaceutically acceptable salt thereof, a polyvinyl pyrrolidone-based compound, a polyethylene glycol-based compound, and sodium dodecyl sulfate; a 39th combination comprising docetaxel or a pharmaceutically acceptable salt thereof, a polyvinyl pyrrolidone-based compound, an alginic acid-based compound, and polygamma glutamic acid; a 40th combination comprising docetaxel or a pharmaceutically acceptable salt thereof, a polyvinyl pyrrolidone-based compound, an alginic acid-based compound, and sodium dodecyl sulfate; a 41st combination comprising docetaxel or a pharmaceutically acceptable salt thereof, a polyvinyl pyrrolidone-based compound, polygamma glutamic acid, and sodium dodecyl sulfate; a 42nd combination comprising docetaxel or a pharmaceutically acceptable salt thereof, a cellulose-based compound, a poloxamer-based compound, and an alginic acid-based compound; a 43rd combination comprising docetaxel or a pharmaceutically acceptable salt thereof, a cellulose-based compound, a poloxamer-based compound, and polygamma glutamic acid; a 44th combination comprising docetaxel or a pharmaceutically acceptable salt thereof, a cellulose-based compound, a poloxamer-based compound, and a polyethylene glycol-based compound; a 45th combination comprising docetaxel or a pharmaceutically acceptable salt thereof, a cellulose-based compound, a poloxamer-based compound, and polygamma glutamic acid; a 46th combination comprising docetaxel or a pharmaceutically acceptable salt thereof, a cellulose-based compound, a poloxamer-based compound, and an alginic acid-based compound; a 47th combination comprising docetaxel or a pharmaceutically acceptable salt thereof, a cellulose-based compound, a poloxamer-based compound, and sodium dodecyl sulfate; a 48th combination comprising docetaxel or a pharmaceutically acceptable salt thereof, a cellulose-based compound, a polyethylene glycol-based compound, and an alginic acid-based compound; a 49th combination comprising docetaxel or a pharmaceutically acceptable salt thereof, a cellulose-based compound, a polyethylene glycol-based compound, and polygamma glutamic acid; a 50th combination comprising docetaxel or a pharmaceutically acceptable salt thereof, a cellulose-based compound, a polyethylene glycol-based compound, and sodium dodecyl sulfate; a 51st combination comprising docetaxel or a pharmaceutically acceptable salt thereof, a cellulose-based compound, an alginic acid-based compound, and sodium dodecyl sulfate; a 52nd combination comprising docetaxel or a pharmaceutically acceptable salt thereof, a cellulose-based compound, an alginic acid-based compound, and polygamma glutamic acid; a 53rd combination comprising docetaxel or a pharmaceutically acceptable salt thereof, a cellulose-based compound, polygamma glutamic acid, and sodium dodecyl sulfate; a 54th combination comprising docetaxel or a pharmaceutically acceptable salt thereof, a poloxamer-based compound, a polyethylene glycol-based compound, and an alginic acid-based compound; a 55th combination comprising docetaxel or a pharmaceutically acceptable salt thereof, a poloxamer-based compound, a polyethylene glycol-based compound, and polygamma glutamic acid; a 56th combination comprising docetaxel or a pharmaceutically acceptable salt thereof, a poloxamer-based compound, a polyethylene glycol-based compound, and an alginic acid-based compound; a 57th combination comprising docetaxel or a pharmaceutically acceptable salt thereof, a poloxamer-based compound, a polyethylene glycol-based compound, and sodium dodecyl sulfate; a 58th combination comprising docetaxel or a pharmaceutically acceptable salt thereof, a poloxamer-based compound, an alginic acid-based compound, and polygamma glutamic acid; a 59th combination comprising docetaxel or a pharmaceutically acceptable salt thereof, a poloxamer-based compound, an alginic acid-based compound, and sodium dodecyl sulfate; a 60th combination comprising docetaxel or a pharmaceutically acceptable salt thereof, a poloxamer-based compound, polygamma glutamic acid, and sodium dodecyl sulfate; a 61st combination comprising docetaxel or a pharmaceutically acceptable salt thereof, a polyvinyl pyrrolidone-based compound, a cellulose-based compound, a poloxamer-based compound, and a polyethylene glycol-based compound; a 62nd combination comprising docetaxel or a pharmaceutically acceptable salt thereof, a polyvinyl pyrrolidone-based compound, a cellulose-based compound, a poloxamer-based compound, and an alginic acid-based compound; a 63rd combination comprising docetaxel or a pharmaceutically acceptable salt thereof, a polyvinyl pyrrolidone-based compound, a cellulose-based compound, a poloxamer-based compound, and polygamma glutamic acid; a 64th combination comprising docetaxel or a pharmaceutically acceptable salt thereof, a polyvinyl pyrrolidone-based compound, a cellulose-based compound, a poloxamer-based compound, and sodium dodecyl sulfate; a 65th combination comprising docetaxel or a pharmaceutically acceptable salt thereof, a polyvinyl pyrrolidone-based compound, a cellulose-based compound, a polyethylene glycol-based compound, and an alginic acid-based compound; a 66th combination comprising docetaxel or a pharmaceutically acceptable salt thereof, a polyvinyl pyrrolidone-based compound, a cellulose-based compound, a polyethylene glycol-based compound, and polygamma glutamic acid; a 67th combination comprising docetaxel or a pharmaceutically acceptable salt thereof, a polyvinyl pyrrolidone-based compound, a cellulose-based compound, a polyethylene glycol-based compound, and sodium dodecyl sulfate; a 68th combination comprising docetaxel or a pharmaceutically acceptable salt thereof, a polyvinyl pyrrolidone-based compound, a cellulose-based compound, an alginic acid-based compound, and polygamma glutamic acid; a 69th combination comprising docetaxel or a pharmaceutically acceptable salt thereof, a polyvinyl pyrrolidone-based compound, a cellulose-based compound, an alginic acid-based compound, and sodium dodecyl sulfate; a 70th combination comprising docetaxel or a pharmaceutically acceptable salt thereof, a polyvinyl pyrrolidone-based compound, a poloxamer-based compound, a polyethylene glycol-based compound, and an alginic acid-based compound; a 71st combination comprising docetaxel or a pharmaceutically acceptable salt thereof, a polyvinyl pyrrolidone-based compound, a poloxamer-based compound, a polyethylene glycol-based compound, and polygamma glutamic acid; a 72nd combination comprising docetaxel or a pharmaceutically acceptable salt thereof, a polyvinyl pyrrolidone-based compound, a poloxamer-based compound, a polyethylene glycol-based compound, and sodium dodecyl sulfate; a 73rd combination comprising docetaxel or a pharmaceutically acceptable salt thereof, a polyvinyl pyrrolidone-based compound, a polyethylene glycol-based compound, an alginic acid-based compound, and polygamma glutamic acid; a 74th combination comprising docetaxel or a pharmaceutically acceptable salt thereof, a polyvinyl pyrrolidone-based compound, a polyethylene glycol-based compound, an alginic acid-based compound, and sodium dodecyl sulfate; a 75th combination comprising docetaxel or a pharmaceutically acceptable salt thereof, a polyvinyl pyrrolidone-based compound, an alginic acid-based compound, polygamma glutamic acid, and sodium dodecyl sulfate; a 76th combination comprising docetaxel or a pharmaceutically acceptable salt thereof, a cellulose-based compound, a poloxamer-based compound, a polyethylene glycol-based compound, and an alginic acid-based compound; a 77th combination comprising docetaxel or a pharmaceutically acceptable salt thereof, a cellulose-based compound, a poloxamer-based compound, a polyethylene glycol-based compound, and polygamma glutamic acid; a 78th combination comprising docetaxel or a pharmaceutically acceptable salt thereof, a cellulose-based compound, a poloxamer-based compound, a polyethylene glycol-based compound, and sodium dodecyl sulfate; a 79th combination comprising docetaxel or a pharmaceutically acceptable salt thereof, a cellulose-based compound, a polyethylene glycol-based compound, an alginic acid-based compound, and polygamma glutamic acid; an 80th combination comprising docetaxel or a pharmaceutically acceptable salt thereof, a cellulose-based compound, an alginic acid-based compound, polygamma glutamic acid, and sodium dodecyl sulfate; an 81st combination comprising docetaxel or a pharmaceutically acceptable salt thereof, a poloxamer-based compound, a polyethylene glycol-based compound, an alginic acid-based compound, and polygamma glutamic acid; an 82nd combination comprising docetaxel or a pharmaceutically acceptable salt thereof, a poloxamer-based compound, a polyethylene glycol-based compound, an alginic acid-based compound, and sodium dodecyl sulfate; an 83rd combination comprising docetaxel or a pharmaceutically acceptable salt thereof, a poloxamer-based compound, an alginic acid-based compound, polygamma glutamic acid, and sodium dodecyl sulfate; an 84th combination comprising docetaxel or a pharmaceutically acceptable salt thereof, a polyvinyl pyrrolidone-based compound, a cellulose-based compound, a poloxamer-based compound, a polyethylene glycol-based compound, and an alginic acid-based compound; an 85th combination comprising docetaxel or a pharmaceutically acceptable salt thereof, a polyvinyl pyrrolidone-based compound, a cellulose-based compound, a poloxamer-based compound, a polyethylene glycol-based compound, and polygamma glutamic acid; an 86th combination comprising docetaxel or a pharmaceutically acceptable salt thereof, a polyvinyl pyrrolidone-based compound, a cellulose-based compound, a poloxamer-based compound, a polyethylene glycol-based compound, and sodium dodecyl sulfate; an 87th combination comprising docetaxel or a pharmaceutically acceptable salt thereof, a polyvinyl pyrrolidone-based compound, a cellulose-based compound, a poloxamer-based compound, an alginic acid-based compound, and polygamma glutamic acid; an 88th combination comprising docetaxel or a pharmaceutically acceptable salt thereof, a polyvinyl pyrrolidone-based compound, a cellulose-based compound, a poloxamer-based compound, an alginic acid-based compound, and sodium dodecyl sulfate; an 89th combination comprising docetaxel or a pharmaceutically acceptable salt thereof, a polyvinyl pyrrolidone-based compound, a cellulose-based compound, a poloxamer-based compound, polygamma glutamic acid, and sodium dodecyl sulfate; a 90th combination comprising docetaxel or a pharmaceutically acceptable salt thereof, a polyvinyl pyrrolidone-based compound, a cellulose-based compound, a polyethylene glycol-based compound, an alginic acid-based compound, and polygamma glutamic acid; a 91st combination comprising docetaxel or a pharmaceutically acceptable salt thereof, a polyvinyl pyrrolidone-based compound, a cellulose-based compound, a polyethylene glycol-based compound, polygamma glutamic acid, and sodium dodecyl sulfate; a 92nd combination comprising docetaxel or a pharmaceutically acceptable salt thereof, a polyvinyl pyrrolidone-based compound, a cellulose-based compound, an alginic acid-based compound, polygamma glutamic acid, and sodium dodecyl sulfate; a 93rd combination comprising docetaxel or a pharmaceutically acceptable salt thereof, a cellulose-based compound, a poloxamer-based compound, a polyethylene glycol-based compound, an alginic acid-based compound, and polygamma glutamic acid; a 94th combination comprising docetaxel or a pharmaceutically acceptable salt thereof, a cellulose-based compound, a poloxamer-based compound, a polyethylene glycol-based compound, an alginic acid-based compound and sodium dodecyl sulfate; a 95th combination comprising docetaxel or a pharmaceutically acceptable salt thereof, a cellulose-based compound, a polyethylene glycol-based compound, an alginic acid-based compound, polygamma glutamic acid, and sodium dodecyl sulfate; a 96th combination comprising docetaxel or a pharmaceutically acceptable salt thereof, a polyvinyl pyrrolidone-based compound, a cellulose-based compound, a poloxamer-based compound, a polyethylene glycol-based compound, an alginic acid-based compound, and polygamma glutamic acid; a 97th combination comprising docetaxel or a pharmaceutically acceptable salt thereof, a polyvinyl pyrrolidone-based compound, a cellulose-based compound, a poloxamer-based compound, a polyethylene glycol-based compound, an alginic acid-based compound, and sodium dodecyl sulfate; a 98th combination comprising docetaxel or a pharmaceutically acceptable salt thereof, a cellulose-based compound, a poloxamer-based compound, a polyethylene glycol-based compound, an alginic acid-based compound, polygamma glutamic acid, and sodium dodecyl sulfate; a 99th combination comprising docetaxel or a pharmaceutically acceptable salt thereof, a polyvinyl pyrrolidone-based compound, a poloxamer-based compound, a polyethylene glycol-based compound, an alginic acid-based compound, polygamma glutamic acid, and sodium dodecyl sulfate; a 99th combination comprising docetaxel or a pharmaceutically acceptable salt thereof, a polyvinyl pyrrolidone-based compound, a poloxamer-based compound, a cellulose-based compound, an alginic acid-based compound, polygamma glutamic acid, and sodium dodecyl sulfate; a 100th combination comprising docetaxel or a pharmaceutically acceptable salt thereof, a polyvinyl pyrrolidone-based compound, a cellulose-based compound, a poloxamer-based compound, a polyethylene glycol-based compound, polygamma glutamic acid, and sodium dodecyl sulfate; a 101st combination comprising docetaxel or a pharmaceutically acceptable salt thereof, a polyvinyl pyrrolidone-based compound, a cellulose-based compound, a poloxamer-based compound, a polyethylene glycol-based compound, an alginic acid-based compound, and sodium dodecyl sulfate; a 102nd combination comprising docetaxel or a pharmaceutically acceptable salt thereof, a polyvinyl pyrrolidone-based compound, a cellulose-based compound, a poloxamer-based compound, a polyethylene glycol-based compound, an alginic acid-based compound, and polygamma glutamic acid; and a 103rd combination comprising docetaxel or a pharmaceutically acceptable salt thereof, a polyvinyl pyrrolidone-based compound, a cellulose-based compound, a poloxamer-based compound, a polyethylene glycol-based compound, an alginic acid-based compound, polygamma glutamic acid, and sodium dodecyl sulfate.

8. The pharmaceutical composition of claim 1, further comprising one or more of magnesium oxide (MgO), bentonite mineral, hydrotalcite, and magnesium hydroxide.

9. The pharmaceutical composition of claim 8, wherein the bentonite mineral contains 90% by weight or more of montmorillonite.

10. The pharmaceutical composition of claim 1, wherein the docetaxel or a pharmaceutically acceptable salt thereof and one or more compounds selected from among a polyvinyl pyrrolidone-based compound, a cellulose-based compound, a poloxamer-based compound, a polyethylene glycol-based compound, and an alginic acid-based compound satisfy the weight ratio range of 1:1 to 1:12.

11. The pharmaceutical composition of claim 1, which is for anti-inflammatory, antiviral, or anticancer purposes.

12. A method for preparing a pharmaceutical composition, the method comprising the steps of: preparing a first dissolved product by dissolving one or more compounds selected from among a polyvinyl pyrrolidone-based compound, a cellulose-based compound, a poloxamer-based compound, a polyethylene glycol-based compound, an alginic acid-based compound, polygamma glutamic acid and sodium dodecyl sulfate in an organic solvent; and
preparing a second dissolved product by adding and stirring docetaxel or a pharmaceutically acceptable salt thereof to the first dissolved product.

13. The method for preparing a pharmaceutical composition of claim 12, further comprising a step for forming powders by evaporating the solvent from the second dissolved product.

14. The method for preparing a pharmaceutical composition of claim 12, further comprising a step for physically mixing one or more selected from among magnesium oxide (MgO), bentonite mineral, hydrotalcite, and magnesium hydroxide with the powders.

15. The method for preparing a pharmaceutical composition of claim 12, further comprising the steps of: preparing a third dissolved product by dispersing one or more of magnesium oxide (MgO), bentonite mineral, hydrotalcite, and magnesium hydroxide in the second dissolved product; and drying the third dissolved product.

16. A method of providing an anti-inflammatory, antiviral, or anticancer effect, the method comprising administering to a subject in need thereof the pharmaceutical composition of claim 1.
